(19) United States
(12) Patent Application Publication
(10) Pub. No.: US 2025/0154267 A2
(43) Pub. Date: May 15, 2025

(54) METHODS FOR TREATING EOSINOPHILIC ESOPHAGITIS BY ADMINISTERING AN IL-4R INHIBITOR

(71) Applicants: REGENERON PHARMACEUTICALS, INC., Tarrytown, NY (US); SANOFI BIOTECHNOLOGY, Paris (FR)

(72) Inventors: Jennifer D. HAMILTON, Ridgefield, CT (US); Jennifer MALONEY, Hastings-on-Hudson, NY (US); Leda MANNENT, Paris (FR); Marcella RUDDY, Wellesley, MA (US)

(21) Appl. No.: 17/327,504

(22) Filed: May 21, 2021

Related U.S. Application Data
(60) Provisional application No. 63/029,085, filed on May 22, 2020.
(60) Provisional application No. 63/066,705, filed on Aug. 17, 2020.
(60) Provisional application No. 63/071,264, filed on Aug. 27, 2020.
(60) Provisional application No. 63/088,147, filed on Oct. 6, 2020.
(60) Provisional application No. 63/121,088, filed on Dec. 3, 2020.
(60) Provisional application No. 63/144,939, filed on Feb. 2, 2021.

(30) Foreign Application Priority Data
Apr. 21, 2021 (EP) 21315068.3

(51) Int. Cl. C07K 16/28; A61K 31/4439; A61K 39/00; A61P 29/00

(52) U.S. Cl. CPC C07K 16/2866; A61K 31/4439; A61P 29/00; A61K 2039/545

(57) ABSTRACT

The present disclosure provides methods for treating, preventing, or ameliorating one or more symptoms of eosinophilic esophagitis in an adolescent or adult subject by administering to the subject one or more doses of an interleukin-4 receptor (IL-4R) inhibitor, such as an anti-IL-4R antibody or antigen-binding fragment thereof.

### CROSS-REFERENCE TO RELATED APPLICATIONS

This application claims priority to United States Provisional Patent Application Nos. 63/029,085, filed May 22, 2020; 63/066,705, filed Aug. 17, 2020; 63/071,264, filed Aug. 27, 2020; 63/088,147, filed Oct. 6, 2020; 63/121,088, filed Dec. 3, 2020; and 63/144,939, filed Feb. 2, 2021; and to European Patent Application No. 21315068.3, filed Apr. 21, 2021; the entire contents of each of which are incorporated by reference.

### SEQUENCE STATEMENT

The instant application contains a Sequence Listing, which has been submitted electronically in ASCII format and is hereby incorporated by reference in its entirety. Said ASCII copy, created on Jul. 16, 2021, is named 40848-0105USU1-SequenceList.txt and is 227 kilobytes in size.

### FIELD OF THE INVENTION

The present disclosure relates to the use of interleukin-4 (IL-4) receptor inhibitors to treat or prevent eosinophilic esophagitis in a subject in need thereof.

### BACKGROUND

Eosinophilic esophagitis (EoE) is a chronic, inflammatory, allergic/immune-mediated disease of the esophagus characterized by local eosinophilic inflammation leading to symptoms of esophageal dysfunction. Although considered a rare disease, the current prevalence is estimated at 22.7 per 100,000 worldwide (Arias, et al., Aliment Pharmaco Ther 2016, 43:3-15) and appears to be on the increase (Dellon, Gastroenterology Clinics of North America 2014, 43:201-218). Eosinophilic esophagitis has been reported in all ages; however, most cases are in children and adults younger than 50 years (see, e.g., Dellon, et al., Clinical Gastroenterology and Hepatology 2014, 12:589-596). Gender differences in EoE have been consistently reported, with males affected 3 to 4 times more often than females, although there are no gender-related differences in the clinical symptoms (see, e.g., Kapel, et al., Gastroenterology 2008, 140:82-90).

The primary clinical manifestations of EoE in both adults and children over 10 years of age are dysphagia and food impaction (Lucendo, et al., United European Gastroenterol J, 2017, 5:335-358). These symptoms lead to substantially impaired quality of life (QOL) (see, DeBrosse, et al., Journal of Allergy and Clinical Immunology 2011, 128:132-138; Falk, Gastrointestinal Endoscopy Clinics of North America 2014, 43:231-242; and Straumann, Gastrointestinal Endoscopy Clinics of North America 2008, 18:99-118). Endoscopic findings are related to the inflammation in the esophagus and consist of fixed or transient concentric rings, longitudinal furrows, white plaques, reduced mucosal vascularity, fragile or crepe-like mucosa, and strictures.

Growing evidence suggests that a Type 2 cytokine-mediated immune response plays an important role in the development of EoE. This is thought to occur by provoking chronic eosinophil, mast cell, T cell, and lymphocyte-induced inflammation via cytokines known to regulate eosinophilic accumulation in the esophagus, such as interleukin (IL)-4, IL-5, IL-13, and eotaxin-1, -2, and -3 (see, e.g., Abonia and Rothenberg, Annual Review of Medicine 2012, 63:421-434; Blanchard, et al., Journal of Clinical Investigation, 2006, 116:536-547; Blanchard, et al., Gastrointestinal Endoscopy Clinics of North America, 2008, 18:133-43; and Mishra, Immunology and Allergy Clinics of North America, 2009, 29:29-40). Consistent with the Type 2-mediated inflammation observed in esophageal tissue, patients with EoE have high rates of comorbid allergic diseases which are also associated with enhanced IL-4 and IL-13 signaling, especially food allergies, atopic dermatitis (AD), asthma, and allergic rhinitis (see, e.g., Assa'ad, Gastrointestinal Endoscopy Clinics of North America, 2008, 18:119-132; and Weinbrand-Goichberg, et al., Immunologic Research, 2013, 56:249-260).

Current therapeutic approaches include chronic dietary elimination, swallowed topical formulation corticosteroids (not approved for the treatment of EoE outside the European Union [EU]), and esophageal dilation. Emergency endoscopy for prolonged and/or painful food impaction is associated with a risk of severe esophageal injury and does not alter the underlying pathogenesis or progression of the disease. Although swallowed topical corticosteroids have been reported in clinical trials to induce partial clinical responses and histologic remission, they are not uniformly effective and can be associated with fungal infections as well as disease recurrence after discontinuation. Accordingly, there remains a need for safe and effective therapies for treating EoE.

### SUMMARY

In one aspect, methods of treating, preventing, or ameliorating at least one symptom of eosinophilic esophagitis (EoE) in a subject≥12 years of age are provided. In some embodiments, the method comprising administering to the subject one or more doses of an interleukin-4 receptor (IL-4R) inhibitor, wherein prior to the onset of treatment the subject has a Dysphagia Symptom Questionnaire (DSQ) score≥10, and wherein the IL-4R inhibitor is an antibody or antigen-binding fragment thereof that binds IL-4Rα and comprises a heavy chain complementarity determining region (HCDR)1 comprising the amino acid sequence of SEQ ID NO:3, an HCDR2 comprising the amino acid sequence of SEQ ID NO:4, an HCDR3 comprising the amino acid sequence of SEQ ID NO:5, a light chain complementarity determining region (LCDR)1 comprising the amino acid sequence of SEQ ID NO:6, an LCDR2 comprising the amino acid sequence of SEQ ID NO:7, and an LCDR3 comprising the amino acid sequence of SEQ ID NO:8.

In some embodiments, the subject is an adult. In some embodiments, the subject is an adolescent≥12 and <18 years of age.

In some embodiments, prior to the onset of treatment the subject has an intraepithelial eosinophilic infiltration peak cell count≥15 eos/hpf as measured by endoscopic biopsy in at least two of the proximal esophageal region, mid esophageal region, and distal esophageal region. In some embodiments, the subject has a history of an average of at least two episodes of dysphagia per week for at least 4 weeks. In some embodiments, the subject is unresponsive or inadequately responsive to treatment with a swallowed topical corticosteroid and/or a proton pump inhibitor (PPI).

In some embodiments, the subject has a concomitant atopic disease. In some embodiments, the concomitant atopic disease is a food allergy, atopic dermatitis, asthma, chronic rhinosinusitis, allergic rhinitis, or allergic conjunctivitis. In some embodiments, the subject has eosinophilic gastroenteritis. In some embodiments, the subject has a current or prior comorbidity that is selected from the group consisting of asthma, atopic dermatitis, hand and food eczema, allergic rhinitis, oral allergy syndrome, and food allergy (e.g., peanut allergy).

In some embodiments, the subject has a concomitant type 2 inflammatory disease. In some embodiments, the subject has one or more of asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), atopic conjunctivitis, vasculitis, cystic fibrosis (CF), chronic obstructive pulmonary disease (COPD), chronic rhinosinusitis with nasal polyps (CRSwNP), aspirin hypersensitivity, non-steroidal anti-inflammatory drug (NSAID) hypersensitivity (e.g., NSAIDs Exacerbated Respiratory Disease, or NSAID-ERD), perennial allergic rhinitis (PAR), atopic dermatitis (AD), chronic eosinophilic pneumonia (CEP), or exercise induced bronchospasm.

In some embodiments, the IL-4R inhibitor comprises a heavy chain variable region (HCVR) comprising the amino acid sequence of SEQ ID NO:1 and a light chain variable region (LCVR) comprising the amino acid sequence of SEQ ID NO:2. In some embodiments, the IL-4R inhibitor comprises a heavy chain comprising the amino acid sequence of SEQ ID NO:9 and a light chain comprising the amino acid sequence of SEQ ID NO:10. In some embodiments, the IL-4R inhibitor is dupilumab or a bioequivalent thereof.

In some embodiments, the IL-4R inhibitor is administered at a dose of about 50 mg to about 600 mg. In some embodiments, the IL-4R inhibitor is administered at a dose of about 300 mg. In some embodiments, the IL-4R inhibitor once a week or once every two weeks.

In some embodiments, the IL-4R inhibitor is administered in combination with a second therapeutic agent or therapy. In some embodiments, the second therapeutic agent or therapy is an IL-1β inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a topical corticosteroid, an oral corticosteroid, a systemic corticosteroid, an inhaled corticosteroid, a glucocorticoid, a PPI, a decongestant, an antihistamine, a non-steroidal anti-inflammatory drug (NSAID), esophagus dilation, allergen removal, or diet management.

In some embodiments, the IL-4R inhibitor is administered in combination with a PPI. In some embodiments, the PPI is administered as a high-dose regimen selected from the group consisting of: omeprazole at a dose of 40 mg QD or 20 mg BID, esomeprazole at a dose of 40 mg QD or 20 mg BID, lansoprazole at a dose of 60 mg QD or 30 mg BID, dexlansoprazole at a dose of 60 mg QD, rabeprazole at a dose of 40 mg QD or 20 mg BID, and pantoprazole at a dose of 80 mg QD or 40 mg BID. In some embodiments, treatment with the IL-4R inhibitor reduces the need for treatment with a PPI.

In some embodiments, treatment with the IL-4R inhibitor normalizes the expression of one or more EoE-associated and/or Type 2 inflammation-associated genes (e.g., normalizes the expression of one or more Type 2 inflammation-associated genes shown in FIG. 3 and/or normalizes the expression of one or more EoE-associated genes shown in FIG. 6). In some embodiments, treatment with the IL-4R inhibitor reduces dysphagia in the subject (e.g., improves a subject's ability to swallow food). In some embodiments, treatment with the IL-4R inhibitor decreases the subject's DSQ score by at least 30%, relative to baseline, after 24 weeks of treatment; and/or decreases the subject's DSQ score by at least 10 points, relative to baseline, after 24 weeks of treatment. In some embodiments, treatment with the IL-4R inhibitor reduces symptoms of dysphagia within about 4 weeks, within about 6 weeks, or within about 8 weeks of starting treatment, relative to a baseline value for the subject. In some embodiments, treatment with the IL-4R inhibitor reduces esophageal intraepithelial eosinophils in the subject. In some embodiments, reduces the subject's peak esophageal intraepithelial eosinophil count by at least 50%, relative to baseline, after 24 weeks of treatment; and/or reduces the subject's peak esophageal intraepithelial eosinophil count to ≤6 eos/hpf after 24 weeks of treatment. In some embodiments, treatment with the IL-4R inhibitor reduces the subject's peak esophageal intraepithelial eosinophil count to ≤1 eos/hpf after 24 weeks of treatment. In some embodiments, treatment with the IL-4R inhibitor improves one or more endoscopic characteristics of the esophagus, e.g., the presence or severity of edema, rings, exudates, furrows, and/or stricture in the proximal and/or distal portion of the esophagus. In some embodiments, treatment with the IL-4R inhibitor reduces the subject's EoE-EREFS score by at least 25%, relative to baseline, after 24 weeks of treatment. In some embodiments, treatment with the IL-4R inhibitor reduces the expression of a biomarker selected from the group consisting of TARC, eotaxin-3, and IgE (e.g., total IgE). In some embodiments, treatment with the IL-4R inhibitor normalizes the expression of a gene that is correlated with a clinical measure of disease severity (e.g., CTSC, CCL26, CCR3, ANO1, and/or SPINK8).

In another aspect, methods of improving the ability to swallow food are provided. In some embodiments, the method comprises:
administering to a subject having eosinophilic esophagitis (EoE) one or more doses of an interleukin-4 receptor (IL-4R) inhibitor, wherein the IL-4R inhibitor is an antibody or antigen-binding fragment thereof that binds IL-4Rα and comprises a heavy chain complementarity determining region (HCDR)1 comprising the amino acid sequence of SEQ ID NO:3, an HCDR2 comprising the amino acid sequence of SEQ ID NO:4, an HCDR3 comprising the amino acid sequence of SEQ ID NO:5, a light chain complementarity determining region (LCDR)1 comprising the amino acid sequence of SEQ ID NO:6, an LCDR2 comprising the amino acid sequence of SEQ ID NO:7, and an LCDR3 comprising the amino acid sequence of SEQ ID NO:8.

In some embodiments, the subject is an adult. In some embodiments, the subject is an adolescent≥12 and <18 years of age.

In some embodiments, prior to the onset of treatment the subject has a Dysphagia Symptom Questionnaire (DSQ) score≥10. In some embodiments, prior to the onset of treatment the subject has an intraepithelial eosinophilic infiltration peak cell count≥15 eos/hpf as measured by endoscopic biopsy in at least two of the proximal esophageal region, mid esophageal region, and distal esophageal region. In some embodiments, the subject has a history of an average of at least two episodes of dysphagia per week for at least 4 weeks. In some embodiments, the subject is unresponsive or inadequately responsive to treatment with a swallowed topical corticosteroid and/or a proton pump inhibitor (PPI).

In some embodiments, the subject has a concomitant atopic disease. In some embodiments, the concomitant atopic disease is a food allergy, atopic dermatitis, asthma, chronic rhinosinusitis, allergic rhinitis, or allergic conjunctivitis. In some embodiments, the subject has eosinophilic gastroenteritis.

In some embodiments, the subject has a concomitant type 2 inflammatory disease. In some embodiments, the subject has one or more of asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), atopic conjunctivitis, vasculitis, cystic fibrosis (CF), chronic obstructive pulmonary disease (COPD), chronic rhinosinusitis with nasal polyps (CRSwNP), aspirin hypersensitivity, non-steroidal anti-inflammatory drug (NSAID) hypersensitivity (e.g., NSAIDs Exacerbated Respiratory Disease, or NSAID-ERD), perennial allergic rhinitis (PAR), atopic dermatitis (AD), chronic eosinophilic pneumonia (CEP), or exercise induced bronchospasm.

In some embodiments, the IL-4R inhibitor comprises a heavy chain variable region (HCVR) comprising the amino acid sequence of SEQ ID NO:1 and a light chain variable region (LCVR) comprising the amino acid sequence of SEQ ID NO:2. In some embodiments, the IL-4R inhibitor comprises a heavy chain comprising the amino acid sequence of SEQ ID NO:9 and a light chain comprising the amino acid sequence of SEQ ID NO:10. In some embodiments, the IL-4R inhibitor is dupilumab or a bioequivalent thereof.

In some embodiments, the IL-4R inhibitor is administered at a dose of about 50 mg to about 600 mg. In some embodiments, the IL-4R inhibitor is administered at a dose of about 300 mg. In some embodiments, the IL-4R inhibitor once a week or once every two weeks.

In some embodiments, the IL-4R inhibitor is administered in combination with a second therapeutic agent or therapy. In some embodiments, the second therapeutic agent or therapy is an IL-1β inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a topical corticosteroid, an oral corticosteroid, a systemic corticosteroid, an inhaled corticosteroid, a glucocorticoid, a PPI, a decongestant, an antihistamine, a non-steroidal anti-inflammatory drug (NSAID), esophagus dilation, allergen removal, or diet management.

In some embodiments, the IL-4R inhibitor is administered in combination with a PPI. In some embodiments, the PPI is administered as a high-dose regimen selected from the group consisting of: omeprazole at a dose of 40 mg QD or 20 mg BID, esomeprazole at a dose of 40 mg QD or 20 mg BID, lansoprazole at a dose of 60 mg QD or 30 mg BID, dexlansoprazole at a dose of 60 mg QD, rabeprazole at a dose of 40 mg QD or 20 mg BID, and pantoprazole at a dose of 80 mg QD or 40 mg BID. In some embodiments, treatment with the IL-4R inhibitor reduces the need for treatment with a PPI.

In some embodiments, treatment with the Il-4R inhibitor:
decreases the subject's DSQ score by at least 30%, relative to baseline, after 24 weeks of treatment;
decreases the subject's DSQ score by at least 10 points, relative to baseline, after 24 weeks of treatment; and/or
improves the subject's Patient Global Impression of Change (PGIC) of Dysphagia score.

In some embodiments, treatment with the Il-4R inhibitor results in the subject scoring a Patient Global Impression of Change (PGIC) of Dysphagia outcome of “very much better” or “moderately better” after 24 weeks of treatment.

In some embodiments, the IL-4R inhibitor is contained within a container selected from the group consisting of a glass vial, a syringe, a pen delivery device, and an autoinjector. In some embodiments, the IL-4R inhibitor is contained within a glass vial. In some embodiments, the IL-4R inhibitor is contained within a syringe. In some embodiments, the IL-4R inhibitor is contained within an autoinjector. In some embodiments, the IL-4R inhibitor is contained within a pen delivery device. In some embodiments, the pen delivery device is prefilled.

Other embodiments will be apparent from a review of the ensuing detailed description.

### BRIEF DESCRIPTION OF THE FIGURES

FIG. 1. Treatment with dupilumab significantly and rapidly reduced dysphagia severity in EoE patients, as measured by change in DSQ total score over a 24-week treatment period. DSQ scores range from 0 to 84, with a lower score indicating less frequent or less severe dysphagia. Placebo=diamond (upper line in graph); dupilumab 300 mg QW=circle (lower line in graph). LS, least squares; SE, standard error. *P<0.05, ** P<0.01; *** P<0.001.

FIGS. 2A and 2B. Treatment with dupilumab reduced endoscopic features of EoE at Week 24, as measured by EREFS score. EREFS score ranges from 0 to 18, with higher score indicating higher severity/presence. FIG. 2A: Absolute change in EREFS total score from baseline. FIG. 2B: Change from baseline to Week 24 in the major features of the EREFS at proximal and distal esophageal regions. ** P<0.01; *** P<0.001. P values are nominal. EREFS, Eosinophilic Esophagitis-Endoscopic Reference Score; LS, least squares; SD, standard deviation; SE, standard error. ^{a} Five patients in the placebo group received rescue treatment; data after rescue treatment were set to missing and their Week 24 data were imputed. Other reasons for missing data include early discontinuation from study in Part A, Week 24 endoscopy was performed after patient took the first dose of Part C study drug, or Week 24 visit was delayed due to COVID-19 pandemic restriction.

FIG. 3. Treatment with dupilumab normalized the Type 2 inflammatory signature (T2INFGS) in esophageal biopsies from adult and adolescent EoE patients from Example 1. Rows indicate the genes of the Type 2 inflammatory signature: IL13RA1, FCER1A, CCL17, ARG1, IL4R, STAT6, CCR4, TSLP, DPP4, SIGLEC8, GATA1, PTGDR2, CCR3, CLC, HRH1, CCL24, ALOX15, CCL26, IL1RL1, HDC, TPSAB1, CMA1, IL25, IL4, GATA3, IL13, IL5, POSTN, CCL13, CCL18, IL33, CCL11, MUC5B, MUCSAC, PTGDS, and FCER2. Each column is the mean gene expression for one patient (up to 3 samples available per patient per timepoint). Gene expression signatures are shown for placebo-treated patients at screening and at treatment Week 24, for dupilumab-treated patients at screening and at treatment Week 24, for healthy controls, and for EoE controls.

FIG. 4. Treatment with dupilumab normalized the 96-gene EoE diagnostic panel (EDPGS) in esophageal biopsies from adult and adolescent EoE patients from Example 1. Rows indicate the genes of the EoE diagnostic panel (Wen, et al., Gastroenterology 2013; 145(6):1289-1299). Each column is the mean gene expression for one patient (up to 3 samples available per patient per timepoint). Gene expression signatures are shown for placebo-treated patients at screening and at treatment Week 24, for dupilumab-treated patients at screening and at treatment Week 24, for healthy controls, and for EoE controls.

FIG. 5. Effect of dupilumab 300 mg QW vs placebo on 1,302 genes with modulated expression (“the DpxOme-EoE” ™) at Week 12 and their enrichment scores (NES_EoE) in each individual sample. Gene expression signatures are shown for placebo-treated patients (n=19) at baseline and at treatment Week 12, for dupilumab-treated patients (n=22) at baseline and at treatment Week 12, for healthy controls, and for EoE controls. Rows indicate the genes of the DpxOme-EoE™.

FIG. 6. The top 30 genes with the greatest change in expression at Week 12 following treatment with dupilumab 300 mg QW vs placebo.

FIG. 7A-7C. The effect of dupilumab 300 mg QW vs. placebo on median change from baseline to Weeks 4, 12, and 24 in biomarkers of type 2 inflammation serum TARC (FIG. 7A), plasma eotaxin-3 (FIG. 7B), and serum total IgE (FIG. 7C) in EoE patients. ***P value between dupilumab and placebo<0.0001. Differences between dupilumab and placebo in change from baseline were analyzed using a rank-based ANCOVA model with baseline measurement as covariate, and stratification factors and the treatment as fixed factors. Values after first rescue treatment used were censored, then the last observation carried forward (LOCF) method was used to impute the missing data at each visit.

### DETAILED DESCRIPTION

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

“Eosinophilic esophagitis” or “EoE,” as used herein, refers to an inflammatory disease characterized by abnormal eosinophilic inflammation within the esophagus and esophageal dysfunction. The primary symptoms of EoE include, but are not limited to, chest and abdominal pain, dysphagia, heartburn, food refusal, vomiting and food impaction. The clinicopathology of EoE is characterized by presence of ridges or trachea-like rings in the esophageal wall and eosinophilic infiltration in the esophageal mucosa. EoE is currently diagnosed by endoscopy of the esophagus with biopsy followed by microscopic and biochemical analysis of the esophageal mucosal lining. EoE may be classified as atopic or non-atopic (see, Mulder, et al., Histopathology 2012, 61:810-822). The present disclosure includes methods to treat both atopic and non-atopic forms of EoE.

As used herein, the term “subject in need thereof” refers to a human or non-human mammal that exhibits one or more symptoms or indications of eosinophilic esophagitis, and/or who has been diagnosed with eosinophilic esophagitis. In certain embodiments, the term includes subjects that show elevated levels of one or more EoE-associated biomarkers (described elsewhere herein) and/or subjects having a gene expression profile that is associated with EoE (“EoE disease transcriptome”). For example, in some embodiments a subject to be treated according to the methods of the disclosure is a subject with elevated levels of IgE, serum TARC, and/or eotaxin-3, a subject having a gene expression profile consistent with the published EoE gene expression signature (Dellon, et al., Clin Transl Gastroenterol 2017, 8(2):e74), or a subject having an altered expression level of one or more genes of the published EoE gene expression signature. As used herein, the terms “subject” and “patient” are used interchangeably.

The term “subject in need thereof” may also include, e.g., subjects who, prior to treatment, exhibit (or have exhibited) one or more indications of EoE such as, e.g., esophageal overexpression of pro-inflammatory mediators such as mast cells, eosinophilic infiltration of the esophagus, thickening of the esophageal wall, dysphagia, food impaction, and chest and abdominal pain and/or an elevated level of a EoE-associated biomarker. The term also includes subjects with elevated peripheral eosinophil counts (e.g., ≥100, ≥150, ≥200, or ≥300 cells/μL) or elevated serum IgE (>150 kU/L).

The term “eosinophilic infiltration” refers to the presence of eosinophils in an organ or tissue including blood, esophagus, stomach, duodenum, and ileum of a subject. In the context of the present disclosure, the term “eosinophilic infiltration” refers to presence of eosinophils in the mucosal lining of a region of the gastro-intestinal tract including, but not limited to, esophagus and stomach. Eosinophilic infiltration is analyzed, for example, in an esophageal tissue biopsy of a subject having EoE. According to some embodiments, “eosinophilic infiltration” refers to the presence of ≥15 eosinophils per high power field in the esophagus, or in two or more of the proximal, mid, and distal regions of the esophagus. The term “high power field” refers to a standard total magnification of 400× by a microscope used to view eosinophils in a tissue, e.g., from the esophagus of a subject. Thus, in some embodiments, a “subject in need thereof” refers to a subject who shows the presence of ≥15 eosinophils (“eos”) per high power field (“hpf”) in the esophagus, e.g., in two or more of the proximal, mid, and distal regions of the esophagus. In certain embodiments, “eosinophilic infiltration” includes infiltration into a tissue by leukocytes, for example, lymphocytes, neutrophils and mast cells. The leukocyte infiltration into, e.g., esophageal tissue can be detected by cell surface markers such as eosinophil-specific markers (e.g., CD11c^{Low/Neg}, SiglecF⁺, F4/80⁺, EMR1⁺, Siglec 8⁺, and MBP2⁺), macrophage-specific markers (e.g., CD11b⁺, F4/80⁺, CD14⁺, EMR1⁺, and CD68⁺), neutrophil-specific markers (e.g., CD11b⁺, Ly6G⁺, Ly6C⁺, CD11b⁺, and CD66b⁺), and T-cell-specific markers (e.g., CD3⁺, CD4⁺, and CD8⁺).

### Therapeutic Methods

In one aspect, methods for treating, preventing, or ameliorating one or more symptoms of eosinophilic esophagitis (EoE) in a subject are provided. In some embodiments, the subject is ≥12 years of age. In some embodiments, the subject is an adult. In some embodiments, the subject is an adolescent≥12 and <18 years of age. In some embodiments, the subject is an adolescent having a body weight>40 kg prior to the onset of treatment.

In some embodiments, a subject to be treated has a history of frequent and/or severe dysphagia. For example, in some embodiments, prior to the onset of treatment the subject has a Dysphagia Symptom Questionnaire (DSQ) score≥10, e.g., a DSQ score≥15, ≥20, ≥25, ≥30, or ≥35. In some embodiments, prior to the onset of treatment the subject experiences an average of at least 2, 3, 4, 5 or more episodes of dysphagia per week. In some embodiments, the subject experiences multiple episodes of dysphagia (e.g., an average of 2 or more episodes of dysphagia) per week for at least 4 weeks, at least 8 weeks, at least 12 weeks, at least 16 weeks, or at least 20 weeks, or for at least 1 month, at least 2 months, at least 3 months, at least 4 months, at least 5 months, at least 6 months, or longer. In some embodiments, prior to the onset of treatment the subject has experienced multiple (e.g., 2 or more) episodes of dysphagia that require liquids, coughing or gagging, vomiting, or medical attention to obtain relief.

In some embodiments, a subject to be treated has an altered level of one or more biomarkers of EoE or has a gene signature profile for EoE-associated genes that is indicative of or consistent with the published gene signature profile for EoE patients. EoE-associated biomarkers and gene expression panels that are diagnostic for EoE are described in the art, e.g., in Sherrill, et al., Genes Immun 2014, 15(6):361-369; Dellon, et al., Clin Trans/Gastroenterol 2017, 8(2):e74; and US Patent Publication No. 2017/0067111. In some embodiments, the subject has an elevated level of eotaxin-3, serum TARC, total IgE, allergen-specific IgE, and/or allergen-specific IgG4. In some embodiments, a subject to be treated has an elevated level of one or more EoE-associated genes such as TNFAIP6, LRRC31, SLC26A4-AS1, ALOX15, CCL26, TGM6, NRXN1, PMCH, SLC26A4, CXCL1, CCR3, TREML2, POSTN, LURAP1L, or CXCL6. In some embodiments, a subject to be treated has a reduced level of one or more EoE-associated genes such as CRTAC1, BC107108, SFTA2, C2orf16, KRTAP3-2, PLNIPRP3, CIDEA, FLG, SLC8A1-AS1, SPINK5, SPINK7, SPINK8, DPCR1, MUC22, CRISP2, DSG1, GYS2, or CRISP3.

In some embodiments, a subject to be treated has, or has had, at least one comorbidity. In some embodiments, the comorbidity is asthma, atopic dermatitis, hand and food eczema, allergic rhinitis, oral allergy syndrome, or food allergy (e.g., peanut allergy).

In some embodiments, a subject to be treated has a concomitant atopic disease. In some embodiments, the concomitant atopic disease is a food allergy, atopic dermatitis, asthma, chronic rhinosinusitis, allergic rhinitis, or allergic conjunctivitis.

In some embodiments, a subject to be treated has, or has had, a concomitant type 2 inflammatory condition. Non-limiting examples of type 2 inflammatory conditions include asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), atopic conjunctivitis, atopic dermatitis, vasculitis, cystic fibrosis (CF), chronic obstructive pulmonary disease (COPD), chronic rhinosinusitis with nasal polyps (CRSwNP), aspirin hypersensitivity, non-steroidal anti-inflammatory drug (NSAID) hypersensitivity (e.g., NSAIDs Exacerbated Respiratory Disease, or NSAID-ERD), perennial allergic rhinitis (PAR), chronic eosinophilic pneumonia (CEP) and exercise induced bronchospasm.

In some embodiments, a subject to be treated is a subject who is susceptible to an allergen, e.g., a subject having a food allergy. For example, in some embodiments, the subject may exhibit one of the following characteristics: (a) is prone to allergic reactions or responses when exposed to one or more allergens; (b) has previously exhibited an allergic response or reaction to one or more allergens; (c) has a known history of allergies; and/or (d) exhibits a sign or symptom of an allergic response or anaphylaxis. As used herein, the phrases “allergic response,” “allergic reaction,” “allergic symptom,” and the like, include one or more signs or symptoms selected from the group consisting of urticaria (e.g., hives), angioedema, rhinitis, asthma, vomiting, sneezing, runny nose, sinus inflammation, watery eyes, wheezing, bronchospasm, reduced peak expiratory flow (PEF), gastrointestinal distress, flushing, swollen lips, swollen tongue, reduced blood pressure, anaphylaxis, and organ dysfunction/failure. An “allergic response,” “allergic reaction,” “allergic symptom,” etc., also includes immunological responses and reactions such as, e.g., increased IgE production, increased allergen-specific immunoglobulin production and/or eosinophilia. In certain embodiments, the subject is allergic to an allergen associated with EoE or that renders the subject susceptible and/or prone to developing EoE. In some embodiments, the allergen is contained within or derived from a food item such as, e.g., dairy products (e.g., cow's milk), egg, wheat, soy, corn, rye, fish, shellfish, peanuts, tree nuts. In some embodiments, the allergen is contained within or derived from a non-food item such as, e.g., dust (e.g., containing dust mite), pollen, insect venom (e.g., venom of bees, wasps, mosquitoes, etc.), mold, animal dander, latex, medication, drugs, ragweed, grass, or birch.

In some embodiments, a subject to be treated exhibits pathology and symptoms that are associated with chronic esophagitis disorders, including gastroesophageal reflux disease (GERD), or has been diagnosed with the chronic esophagitis disorder. In some embodiments, a subject to be treated exhibits pathology and symptoms that are associated with eosinophilic gastroenteritis or has been diagnosed with eosinophilic gastroenteritis.

In some embodiments, a subject to be treated is a subject who is non-responsive, inadequately responsive, or resistant to one or more of the current standard-of-care therapies for EoE (e.g., food-elimination diets, swallowed topical corticosteroids, glucocorticoids, PPI therapy such as high-dose PPI regimens, or esophageal dilation). In some embodiments, the subject is on a high-dose PPI regimen at the onset of treatment with the IL-4R inhibitor. In some embodiments, the subject has had one or more esophageal dilations.

### Anti-IL-4Rα Antibodies and Antigen-Binding Fragments Thereof

According to certain exemplary embodiments of the present disclosure, the IL-4R inhibitor is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (iX) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (Xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein, et al., 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc. [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor, et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Rα. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4Rα, as used in the context of the present disclosure, includes antibodies that bind IL-4Rα or a portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay. An isolated antibody that specifically binds human IL-4Rα may, however, have cross-reactivity to other antigens, such as IL-4Rα molecules from other (non-human) species.

In certain exemplary embodiments, the IL-4R antagonist is an anti-IL-4Rα antibody, or antigen-binding fragment thereof comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693. In certain exemplary embodiments, the anti-IL-4Rα antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. In some embodiments, the anti-IL-4Rα antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3, the HCDR2 comprises the amino acid sequence of SEQ ID NO: 4, the HCDR3 comprises the amino acid sequence of SEQ ID NO: 5, the LCDR1 comprises the amino acid sequence of SEQ ID NO: 6, the LCDR2 comprises the amino acid sequence of SEQ ID NO: 7, and the LCDR3 comprises the amino acid sequence of SEQ ID NO: 8.

In some embodiments, the anti-IL-4R antibody or antigen-binding fragment thereof comprises the HCDR1, HCDR2, HCDR3, LCDR1, LCDR2, and LCDR3 of SEQ ID NOs:3, 4, 5, 6, 7, and 8, respectively, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO: 1 and an LCVR comprising SEQ ID NO: 2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren, et al., 2010, Am J Respir Crit Care Med., 181(8):788-796), or MEDI 9314, or any of the anti-IL-4Rα antibodies as set forth in U.S. Pat. No. 7,186,809, 7,605,237, 7,638,606, 8,092,804, 8,679,487, or 8,877,189, 10,774,141, or International Patent Publication No. WO2020/096381, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 10 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-VL-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55).

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6NL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11NL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16NL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-VL), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL).

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171).

In some embodiments, an anti-IL-4Rα antibody used in the methods of the present disclosure can have pH-dependent binding characteristics. For example, an anti-IL-4Rα antibody for use in the methods of the present disclosure may exhibit reduced binding to IL-4Rα at acidic pH as compared to neutral pH. Alternatively, an anti-IL-4Rα antibody of the disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2 and HCDR3) contained within a heavy chain variable region (HCVR) having an amino acid sequence of SEQ ID NO: 1, and the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani, et al., J. Mol. Biol. 273:927-948 (1997); and Martin, et al., Proc. Natl. Acad. Sci. USA 86:9268-9272 (1989). Public databases are also available for identifying CDR sequences within an antibody.

### Pharmaceutical Compositions

In one aspect, the present disclosure provides methods that comprise administering an IL-4R inhibitor to a subject, wherein the IL-4R inhibitor (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a patient according to the methods of the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti, et al., 1991, Pharmaceut. Res. 8:1351). Specific exemplary doses of anti-IL4R antibodies, and administration regimens involving the same, that can be used in the context of the present disclosure are disclosed elsewhere herein.

Various delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu, et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, a pharmaceutical composition of the present disclosure is contained within a container. Thus, in another aspect, containers comprising a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Examples of disposable pen delivery devices having applications in subcutaneous delivery of a pharmaceutical composition of the present disclosure include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present disclosure are disclosed, e.g., in U.S. Pat. No. 8,945,559.

### Dosage and Administration Regimens

Typically, an amount of IL-4R inhibitor (e.g., an anti-IL-4R antibody as disclosed herein) that is administered to a subject according to the methods disclosed herein is a therapeutically effective amount. As used herein, the phrase “therapeutically effective amount” means an amount of IL-4R inhibitor that results in one or more of: (a) a reduction in the severity or duration of a symptom of eosinophilic esophagitis; (b) a reduction in the number of eosinophils in esophagus; (c) increase in esophagus distensibility; (d) reduction in episodes of dysphagia or the intensity of dysphagia; (e) normalization of one or more EoE-associated biomarkers or gene expression signatures; and/or (f) a reduction in the use or need for concomitant or rescue treatment with another agent (e.g., reduced or eliminated use of systemic and/or swallowed topical corticosteroids, PPIs, etc.).

In the case of an anti-IL-4R antibody, a therapeutically effective amount can be from about 0.05 mg to about 600 mg, about 50 mg to about 600 mg, or about 50 mg to about 300 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In certain embodiments, 75 mg, 100 mg, 150 mg, 200 mg, or 300 mg of an anti-IL-4R antibody is administered to a subject.

The amount of IL-4R inhibitor (e.g., anti-IL-4R antibody) contained within the individual doses may be expressed in terms of milligrams of active agent (e.g., antibody) per kilogram of patient body weight (i.e., mg/kg). For example, the IL-4R inhibitor may be administered to a patient at a dose of about 0.0001 to about 10 mg/kg of patient body weight, e.g., from about 1 mg/kg to about 10 mg/kg, or about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, the IL-4R inhibitor or pharmaceutical composition comprising an IL-4R inhibitor is administered to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments involving the administration of a pharmaceutical composition comprising an anti-IL-4R antibody, once a week dosing at an amount of about 50 mg to about 600 mg, e.g., about 75 mg, 150 mg, 200 mg, or 300 mg, can be employed.

In some embodiments, multiple doses of an IL-4R inhibitor are administered to a subject over a defined time course. In some embodiments, the methods of the present disclosure comprise sequentially administering to a subject multiple doses of an IL-4R inhibitor. As used herein, “sequentially administering” means that each dose of IL-4R inhibitor is administered to the subject at a different point in time, e.g., on different days separated by a predetermined interval (e.g., hours, days, weeks or months). In some embodiments, the methods of the disclosure comprise sequentially administering to the patient a single initial dose of an IL-4R inhibitor, followed by one or more secondary doses of the IL-4R inhibitor, and optionally followed by one or more tertiary doses of the IL-4R inhibitor.

The terms “initial dose,” “secondary dose(s),” and “tertiary dose(s)” refer to the temporal sequence of administration of the IL-4R inhibitor. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R inhibitor, but generally may differ from one another in terms of frequency of administration. In certain embodiments, however, the amount of IL-4R inhibitor contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an IL-4R inhibitor may be administered to a subject at a loading dose of about 200 mg, 400 mg, or about 600 mg followed by one or more maintenance doses of about 75 mg to about 300 mg. In one embodiment, the initial dose and the one or more secondary doses each include 50 mg to 600 mg of the IL-4R inhibitor, e.g., 100 mg, 150 mg, 200 mg, 250 mg, 300 mg, 400 mg, 500 mg, or 600 mg of the IL-4R inhibitor. In some embodiments, the initial dose and the one or more secondary doses each contain the same amount of the IL-4R inhibitor. In other embodiments, the initial dose comprises a first amount of the IL-4R inhibitor, and the one or more secondary doses each comprise a second amount of the IL-4R inhibitor. For example, the first amount of the IL-4R inhibitor can be 1.5×, 2×, 2.5×, 3×, 3.5×, 4× or 5× or more than the second amount of the IL-4R inhibitor. In some embodiments, a subject is administered an IL-4R inhibitor (e.g., one or more doses from about 50 mg to about 600 mg, e.g., about 50 mg, about 75 mg, about 100 mg, about 150 mg, about 200 mg, about 250 mg, about 300 mg, about 350 mg, about 400 mg, about 450 mg, about 500 mg, about 550 mg, or about mg) without a loading dose.

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of IL-4R inhibitor which is administered to a patient prior to the administration of the very next dose in the sequence with no intervening doses.

The methods of the disclosure may comprise administering to a patient any number of secondary and/or tertiary doses of an IL-4R inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 to 2 weeks after the immediately preceding dose. Similarly, in some embodiments involving multiple tertiary doses, each tertiary dose is administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, for a subject having eosinophilic esophagitis who is ≥12 years of age, a therapeutically effective amount of an IL-4R inhibitor (e.g., an anti-IL-4R antibody as disclosed herein) comprises an initial (loading) dose followed by one or more secondary (maintenance) doses, wherein the initial dose of the IL-4R inhibitor (e.g., anti-IL-4R antibody) comprises 600 mg and each secondary dose of the IL-4R inhibitor (e.g., anti-IL-4R antibody) comprises 300 mg, administered every week (QW).

In some embodiments, for a subject having eosinophilic esophagitis who is ≥12 years of age, a therapeutically effective amount of an IL-4R inhibitor (e.g., an anti-IL-4R antibody) comprises an initial (loading) dose followed by one or more secondary (maintenance) doses, wherein the initial dose of the IL-4R inhibitor (e.g., anti-IL-4R antibody) comprises 600 mg and each secondary dose of the IL-4R inhibitor (e.g., anti-IL-4R antibody) comprises 300 mg, administered every two weeks (Q2W).

In some embodiments, for a subject having eosinophilic esophagitis who is ≥12 years of age, a therapeutically effective amount of an IL-4R inhibitor (e.g., an anti-IL-4R antibody) comprises a dose of the IL-4R inhibitor (e.g., anti-IL-4R antibody) comprising 300 mg administered every week (QW).

In some embodiments, for a subject having eosinophilic esophagitis who is ≥12 years of age, a therapeutically effective amount of an IL-4R inhibitor (e.g., an anti-IL-4R antibody) comprises a dose of the IL-4R inhibitor (e.g., anti-IL-4R antibody) comprising 300 mg administered every two weeks (Q2W).

### EoE-Related Parameters

In some embodiments, the therapeutic methods disclosed herein result in an improvement in one or more endpoints or EoE-related parameters that are used to assess the presence or severity of EoE in a subject. Examples of EoE-related parameters include, but are not limited to: (a) change (e.g., reduction) in frequency and/or intensity of dysphagia, e.g., as measured using the Dysphagia Symptom Questionnaire (DSQ), the Straumann Dysphagia Instrument (SDI), the Patient Global Impression of Change (PGIC) or Patient Global Impression of Severity (PGIS) of Dysphagia; (b) change (e.g., reduction) in esophageal intraepithelial eosinophil count; (c) change in one or more esophageal characteristics, e.g., the absence, presence, or severity of edema, rings, exudates, furrows, and/or stricture, e.g., as measured using the EoE-EREFS; (d) change (e.g., increase) in esophageal distensibility, e.g., as measured using an endoluminal functional lumen imaging probe (EndoFLIP); (e) change in the severity and/or extent of histologic features in the esophagus, e.g., as measured using the Eosinophilic Esophagitis Histological Scoring System (EoE-HSS); (f) change (e.g., normalization) in the levels of one or more EoE-associated biomarkers or in the EoE gene expression signature; or (g) change in the frequency and/or severity of other symptoms of EoE, e.g., as measured using the Eosinophilic Esophagitis Impact Questionnaire (EoE-IQ), the EoE Symptom Questionnaire, the Eosinophilic Esophagitis Activity Index (EEsAI), the Adult Eosinophilic Quality of Life (EoE-QQL-A), or the European Quality of Life 5-Dimensional Scale (EQ-5D). Methods for assessing these EoE-related parameters are described in the Examples section below and are also disclosed in WO 2019/028367, incorporated by reference herein.

To determine whether an EoE-related parameter has “improved,” the parameter is quantified at baseline and at one or more timepoints after administration of the IL-4R inhibitor. For example, an EoE-related parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with a pharmaceutical composition of the present disclosure. The difference between the value of the parameter at a particular timepoint following initiation of treatment and the value of the parameter at baseline is used to establish whether there has been an improvement in the EoE-related parameter.

In some embodiments, treatment of a subject with an IL-4R inhibitor (e.g., an anti-IL-4R antibody) according to the methods disclosed herein results in an improvement in symptoms of dysphagia. In some embodiments, treatment results in a change (e.g., reduction) in the frequency and/or intensity of dysphagia in a subject. In some embodiments, treatment results in a reduction in the frequency of episodes of dysphagia per week, e.g., a decrease of at least about 10%, 15%, 20%, 25%, 30%, 35%, 40%, 45%, 50% or more relative to baseline (e.g., the subject's average frequency of episodes of dysphagia per week prior to the onset of treatment). In some embodiments, treatment results in an improvement in DSQ score. The DSQ is a validated PRO that has been used in clinical studies to measure the frequency and intensity of dysphagia (Hudgens, et al., J Patient Rep Outcomes 2017, 1(1):3, doi:10.1186/s41687-017-0006-5). The DSQ uses a daily recall period and comprises 3 questions on the presence and severity of EoE dysphagia. Typically in the DSQ, the patient responds to at least questions 1 and 2 and is required to have eaten solid foods (‘Yes’ to question 1: “Since you woke up this morning, did you eat solid food?”) in order to proceed with the questionnaire. If a patient answers “No” to question 1, the remaining items on the DSQ are not scored. Patients who respond “No” to question 2 (“Since you woke up this morning, has food gone down slowly or been stuck in your throat?”) are given a score of zero, and do not go on to answer question 3 (the diary is recorded as completed for that day). Those who respond ‘Yes’ to questions 1 and 2 move on to question 3, which is scored on a 5-point scale that infers severity of dysphagia based on the patient's action to alleviate symptoms, ranging from no action to seeking medical attention. The DSQ scoring algorithm is therefore constructed from the responses to questions 2 and 3, to ensure that the final score is driven by the frequency and severity of dysphagia. To calculate the DSQ score, a minimum of 8 diary entries is required for each 14-day period to derive a standardized total score based on the cumulative scores through 14 days. In some embodiments, the DSQ is a modified DSQ in which for patients who respond “No” to Question 1 (“Since you woke up this morning, did you eat solid food?”), a follow-up question probes if patients avoided solid food due to their problems with swallowing solid food. DSQ scores can theoretically range from 0 to 84, with a lower score indicating less-frequent or less-severe dysphagia. In some embodiments, treatment with an IL-4R inhibitor results in a decrease in DSQ score relative to baseline (e.g., a subject's DSQ score prior to the onset of treatment). In some embodiments, treatment with an IL-4R inhibitor results in a decrease in DSQ score of at least 3, 4, 5, 6, 7, 8, 9, 10, 11, 12, 13, 14, 15, 16, 17, 18, 19, 20 or more points relative to baseline. In some embodiments, treatment with an IL-4R inhibitor results in a decrease in DSQ score of at least 10%, 15%, 20%, 25%, 30%, 35%, 40%, 45%, 50% or more relative to baseline. In some embodiments, the change in DSQ score is measured at day 8, 15, 22, 25, 29, 36, 43, 50, 57, 64, 71, 85 or later following administration of the IL-4R inhibitor, or after 24 weeks of treatment with the IL-4R inhibitor. In some embodiments, treatment with an IL-4R inhibitor reduces symptoms of dysphagia in a subject (e.g., as measured by change in absolute DSQ score or percentage decrease in DSQ score relative to a baseline value for the subject) within about 4 weeks, 5 weeks, 6 weeks, 7 weeks, 8 weeks, 9 weeks, or 10 weeks of starting treatment with the IL-4R inhibitor.

Changes in symptoms of dysphagia can also be assessed using the Patient Global Impression of Change (PGIC) of Dysphagia. The PGIC is a one-item questionnaire that asks patients to provide the overall self-assessment of change of difficulty swallowing food on a 7-point scale (very much better; moderately better; a little better; no change; a little worse; moderately worse; or very much worse). In some embodiments, treatment with an IL-4R inhibitor results in a decrease in PGIC score relative to baseline (e.g., a subject's PGIC score prior to the onset of treatment). In some embodiments, treatment with an IL-4R inhibitor results in a decrease in PGIC score of at least 1, 2, 3 or more points relative to baseline. In some embodiments, treatment with an IL-4R inhibitor results in a PGIC rating of “very much better” or “moderately better.” In some embodiments, treatment with an IL-4R inhibitor reduces symptoms of dysphagia in a subject (e.g., as measured by improvement in in PGIC score relative to a baseline value for the subject) within about 4 weeks, 5 weeks, 6 weeks, 7 weeks, 8 weeks, 9 weeks, or 10 weeks of starting treatment with the IL-4R inhibitor.

In some embodiments, treatment results in an improvement in SDI score. The SDI is a non-validated patient reported outcome (PRO) that has been used in clinical studies to determine the frequency and intensity of dysphagia (Straumann 2010). The SDI has a 1-week recall period. Frequency of dysphagia events is graded on a 5-point scale: 0=none, 1=once per week, 2=several times per week, 3=once per day, and 4=several times per day, and intensity of dysphagia events is graded on a 6-point scale: 0=swallowing unrestricted, 1=slight sensation of resistance, 2=slight retching with delayed passage, 3=short period of obstruction necessitating intervention (e.g., drinking, breathing), 4=longer-lasting period obstruction only removable by vomiting, and 5=long-lasting complete obstruction requiring endoscopic intervention. The total SDI score ranges from 0 to 9. In some embodiments, treatment with an IL-4R inhibitor results in a decrease in SDI score of 1, 2, 3, 4, 5, 6 or more points relative to baseline (e.g., a subject's SDI score prior to the onset of treatment). In some embodiments, treatment with an IL-4R inhibitor results in a decrease in SDI score of at least 3 points relative to baseline. In some embodiments, treatment with an IL-4R inhibitor results in a decrease in SDI score of at least 10%, 15%, 20%, 25%, 30%, 35%, 40%, 45%, 50% or more relative to baseline. In some embodiments, the change in SDI score is measured at day 8, 15, 22, 25, 29, 36, 43, 50, 57, 64, 71, 85 or later following administration of the IL-4R inhibitor, or after 24 weeks of treatment with the IL-4R inhibitor.

In some embodiments, treatment of a subject with an IL-4R inhibitor (e.g., an anti-IL-4R antibody) results in an improvement (e.g., reduction) in peak esophageal intraepithelial eosinophil count. “Peak esophageal intraepithelial eosinophil count” refers to the number of eosinophils contained within one high power field (hpf). In some embodiments, treatment with an IL-4R inhibitor results in a decrease in peak esophageal intraepithelial eosinophil count relative to baseline (e.g., a subject's peak count prior to the onset of treatment). In some embodiments, treatment with an IL-4R inhibitor results in peak esophageal intraepithelial eosinophil count of at least 10%, 15%, 20%, 25%, 30%, 35%, 40%, 45%, 50% or more relative to baseline. In some embodiments, treatment with an IL-4R inhibitor results in a decrease in peak esophageal intraepithelial eosinophil count to less than 10 eos/hpf or less than 6 eos/hpf. In some embodiments, treatment with an IL-4R inhibitor results in a decrease in peak esophageal intraepithelial eosinophil count to ≤6 eos/hpf, ≤5 eos/hpf, ≤4 eos/hpf, ≤3 eos/hpf, ≤2 eos/hpf, or ≤1 eos/hpf. In some embodiments, the change in peak esophageal intraepithelial eosinophil count measured at day 8, 15, 22, 25, 29, 36, 43, 50, 57, 64, 71, 85 or later following administration of the IL-4R inhibitor, or after 24 weeks of treatment with the IL-4R inhibitor.

In some embodiments, treatment of a subject with an IL-4R inhibitor (e.g., an anti-IL-4R antibody) results in an improvement in one or more endoscopic features of EoE. In some embodiments, treatment of a subject with an IL-4R inhibitor (e.g., an anti-IL-4R antibody) results in an improvement in EoE-EREFS score. The EoE-EREFS (edema, rings, exudates, furrows, strictures) is validated scoring system for inflammatory and remodeling features of disease that is used to measure the endoscopically identified EoE esophageal mucosal inflammatory and remodeling features (Hirano 2014). This instrument includes a total of 17 items related to the presence and severity of esophageal features. The specific esophageal features include: rings (concentric rings around esophagus—absent, mild, moderate, severe, not applicable); strictures (narrowing of the esophagus—yes, no, not applicable); diameter of the stricture (if applicable); exudates (refer to white plaques—absent, mild, severe), furrows (vertical lines down the esophagus—absent, present); edema (loss of vascular markings of the mucosa—absent, present); crepe paper esophagus (absent, present); overall general appearance incorporating all endoscopically identified EoE findings (i.e., fixed rings, strictures, whitish exudates, furrowing, edema, and crepe paper mucosa). In addition, mucosal changes associated with gastroesophageal reflux disease are recorded using the Los Angeles classification system for erosions (No Erosions or LA Classification A, B, C, D). In some embodiments, treatment with an IL-4R inhibitor results in a decrease in EoE-EREFS score of at least 10%, 15%, 20%, 25%, 30%, 35%, 40%, 45%, 50% or more relative to baseline (e.g., a subject's EoE-EREFS score prior to the onset of treatment). In some embodiments, the change in EoE-EREFS score is measured at day 8, 15, 22, 25, 29, 36, 43, 50, 57, 64, 71, 85 or later following administration of the IL-4R inhibitor, or after 24 weeks of treatment with the IL-4R inhibitor.

In some embodiments, treatment of a subject with an IL-4R inhibitor (e.g., an anti-IL-4R antibody) results in an improvement in one or more histological features of EoE. In some embodiments, treatment of a subject with an IL-4R inhibitor (e.g., an anti-IL-4R antibody) results in an improvement in EoE-HSS score. The EoE-HSS is a validated instrument that generates separate severity (grade) and extent (stage) disease scores. The score is used to measure 8 histologic features (parameters) of EoE from 3 different regions (proximal, mid and distal) of the esophagus (Collins, et al. 2017). The 8 parameters include: eosinophil density, basal zone hyperplasia, eosinophil abscesses, eosinophil surface layering, dilated intercellular spaces, surface epithelial alteration, dyskeratotic cells, and lamina propria fibrosis. A scale of 0-3 is used for each parameter, both grade and stage (with 0 being least inflamed, normal). In some embodiments, treatment with an IL-4R inhibitor results in a decrease in EoE-HSS score of at least 10%, 15%, 20%, 25%, 30%, 35%, 40%, 45%, 50% or more relative to baseline (e.g., a subject's EoE-HSS score prior to the onset of treatment). In some embodiments, treatment with an IL-4R inhibitor results in a decrease in EoE-HSS composite score, grade score, and/or stage score. In some embodiments, the change in EoE-HSS score is measured at day 8, 15, 22, 25, 29, 36, 43, 50, 57, 64, 71, 85 or later following administration of the IL-4R inhibitor, or after 24 weeks of treatment with the IL-4R inhibitor.

In some embodiments, treatment of a subject with an IL-4R inhibitor (e.g., an anti-IL-4R antibody) results in an improvement in esophageal distensibility. In some embodiments, esophageal distensibility is assessed using an endoluminal functional lumen imaging probe (EndoFLIP, Medtronic, USA) to measure the diameter of the esophageal lumen and pressure. The EndoFLIP device is a catheter-based procedure that measures the cross-sectional area at multiple sites along the esophagus with simultaneous intraluminal pressure recordings during volumetric distension of the esophagus. The analyses of cross-sectional area versus pressure relationships of the esophagus allow for determination of esophageal compliance as well as the distensibility plateau (DP). The DP has been shown to be significantly reduced in patients with EoE compared to healthy controls (Kwiatek 2011). In some embodiments, treatment with an IL-4R inhibitor results in an increase in esophageal distensibility (e.g., as measured by DP) of at least 10%, 15%, 20%, 25%, 30%, 35%, 40%, 45%, 50% or more relative to baseline (e.g., a subject's esophageal distensibility or DP prior to the onset of treatment). In some embodiments, treatment with an IL-4R inhibitor results in an increase in DP of at least 0.5 mm, 1 mm, 1.5 mm, or more. In some embodiments, the change in esophageal distensibility is measured at day 8, 15, 22, 25, 29, 36, 43, 50, 57, 64, 71, 85 or later following administration of the IL-4R inhibitor, or after 24 weeks of treatment with the IL-4R inhibitor.

In some embodiments, treatment of a subject with an IL-4R inhibitor (e.g., an anti-IL-4R antibody) according to the methods disclosed herein results in an improvement in health-related quality of life. In some embodiments, treatment results in an improvement in EoE-IQ score. The EoE-IQ assesses the impact of EoE on a scale of 1 to 5; higher scores indicate greater health-related QoL impairment. In some embodiments, treatment with an IL-4R inhibitor results in a decrease in EoE-IQ score of at least 10%, 15%, 20%, 25%, 30%, 35%, 40%, 45%, 50% or more relative to baseline. In some embodiments, the change in EoE-IQ score is measured at day 8, 15, 22, 25, 29, 36, 43, 50, 57, 64, 71, 85 or later following administration of the IL-4R inhibitor, or after 24 weeks of treatment with the IL-4R inhibitor. In some embodiments, treatment with an IL-4R inhibitor improves health-related quality of life in a subject (e.g., as measured by change in absolute EoE-IQ score or percentage decrease in EoE-IQ score relative to a baseline value for the subject) within about 4 weeks, 5 weeks, 6 weeks, 7 weeks, 8 weeks, 9 weeks, or 10 weeks of starting treatment with the IL-4R inhibitor.

In some embodiments, treatment of a subject with an IL-4R inhibitor (e.g., an anti-IL-4R antibody) according to the methods disclosed herein results in an improvement in symptoms other than dysphagia. In some embodiments, treatment results in an improvement in EoE-SQ-Frequency score. The EoE-SQ-Frequency assesses symptoms other than dysphagia on a scale of 5 to 25; higher scores indicate higher symptom burden. In some embodiments, treatment with an IL-4R inhibitor results in a decrease in EoE-SQ-Frequency score of at least 1, 2, 3, 4, 5, 6 or more points relative to baseline. In some embodiments, treatment with an IL-4R inhibitor results in a decrease in EoE-SQ-Frequency score of at least 10%, 15%, 20%, 25%, 30%, 35%, 40%, 45%, 50% or more relative to baseline. In some embodiments, the change in EoE-SQ-Frequency score is measured at day 8, 15, 22, 25, 29, 36, 43, 50, 57, 64, 71, 85 or later following administration of the IL-4R inhibitor, or after 24 weeks of treatment with the IL-4R inhibitor. In some embodiments, treatment with an IL-4R inhibitor improves symptoms other than dysphagia in a subject (e.g., as measured by change in absolute EoE-SQ-Frequency score or percentage decrease in EoE-SQ-Frequency score relative to a baseline value for the subject) within about 4 weeks, 5 weeks, 6 weeks, 7 weeks, 8 weeks, 9 weeks, or 10 weeks of starting treatment with the IL-4R inhibitor.

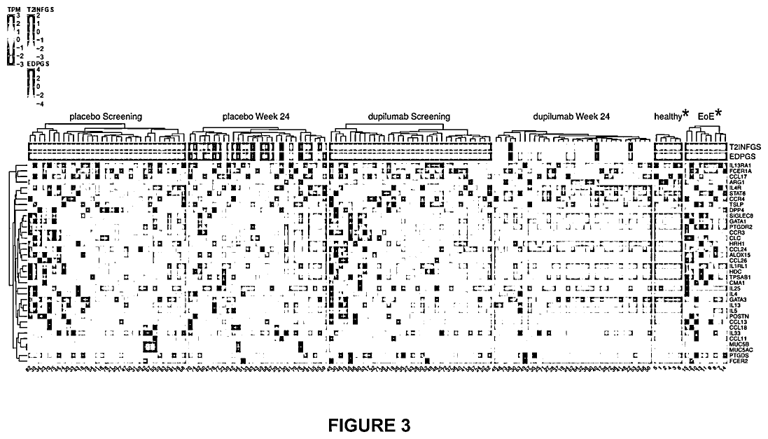

In some embodiments, treatment of a subject with an IL-4R inhibitor (e.g., an anti-IL-4R antibody) results in a normalization of one or more EoE-associated biomarkers, an EoE gene signature, a Type 2 inflammation gene signature, and/or a Normalized Enrichment Score (NES) calculated for a set of EoE-associated genes. In some embodiments, treatment of a subject with an IL-4R inhibitor suppresses an EoE gene signature, a Type 2 inflammation gene signature, and/or an NES calculated for a set of EoE-associated or Type 2 inflammation genes. As used herein, the term “EoE-associated biomarker” refers to a biological response, cell type, parameter, protein, polypeptide, enzyme, enzyme activity, metabolite, nucleic acid, carbohydrate, or other biomolecule which is present or detectable in an EoE patient at a level or amount that is different from (e.g., greater than or less than) the level or amount of the marker present or detectable in a non-EoE patient. In some embodiments, the EoE-associated biomarker is a gene associated with fibrosis, tissue remodeling, or epithelial barrier function. Exemplary EoE-associated biomarkers include, but are not limited to, e.g., esophagus eosinophils, eotaxin-3 (CCL26), periostin (POSTN), serum IgE (total and allergen-specific), serum IgG (total and allergen-specific), arachidonate 15-lipoxygenase (ALOX15), IL-13, IL-5, serum thymus and activation regulated chemokine (TARC; CCL17), thymic stromal lymphopoietin (TSLP), serum eosinophilic cationic protein (ECP), collagen genes (e.g., COL4A3, COL4A4, COL4A6, COL8A2, COL14A1, and COL21A1), calpain 14, desmoglein-1 (DSG1), filaggrin (FLG), signal transducer and activator of transcription 6 (STAT6), serine peptidase inhibitor Kazal-type 5 (SPINK5), SPINK7, SPINK8, interleukin 4 receptor (IL-4R), eosinophil-associated genes (e.g., CLC and SIGLEC8), anoctamin-1 (ANO1), cathepsin C (CTSC), C-C chemokine receptor type 3 (CCR3), and eosinophil-derived neurotoxin (EDN). The term “EoE gene signature” refers to a differential gene expression profile of esophageal biopsies of EoE patients compared to healthy controls, and is also referred to as an “EoE disease transcriptome” (Sherrill, 2014). In some embodiments, an EoE gene signature is a smaller gene set of the published EoE disease transcriptome, such as the EoE diagnostic panel (EDP, clinically available as EoGenuis™, Inform Diagnostics, USA). A “Type 2 inflammation gene signature” refers to a transcriptome for a set of genes associated with type 2 inflammation. Exemplary Type 2 inflammation-associated genes include, but are not limited to, CCL26, ALOX15, CCR3, and IL1RL1. An exemplary gene list for a Type 2 inflammation gene signature is shown in FIG. 3. A Normalized Enrichment Score (NES) reflects the degree to which the activity level of a set of transcripts is overrepresented at the extremes (top or bottom) of the entire ranked list of transcripts within a sample and is normalized by accounting for the number of transcripts in the set (Subramanian, 2005) (Barbie, 2009).

In some embodiments, the EoE-associated biomarkers, EoE gene signature, Type 2 inflammation gene signature, and/or NES are determined using a tissue sample from the subject (e.g., esophageal pinch biopsy samples from the proximal, mid, and/or distal regions). In some embodiments, treatment of a subject with an IL-4R inhibitor results in a normalization of one or more EoE-associated biomarkers, an EoE gene signature, a Type 2 inflammation gene signature, and/or an NES, relative to baseline (e.g., a subject's level of expression of the EoE-associated biomarker(s), EoE gene signature, or NES prior to the onset of treatment), e.g., as measured at day 8, 15, 22, 25, 29, 36, 43, 50, 57, 64, 71, 85 or later following administration of the IL-4R inhibitor, or after 24 weeks of treatment with the IL-4R inhibitor. In some embodiments, treatment of a subject with an IL-4R inhibitor suppresses a NES for one or more EoE-associated biomarkers, an EoE gene signature, or a Type 2 inflammation gene signature, relative to baseline (e.g., a subject's NES prior to the onset of treatment), e.g., as measured at day 8, 15, 22, 25, 29, 36, 43, 50, 57, 64, 71, 85 or later following administration of the IL-4R inhibitor, or after 24 weeks of treatment with the IL-4R inhibitor.

In some embodiments, treatment of a subject with an IL-4R inhibitor (e.g., an anti-IL-4R antibody) results in a normalization of a Type 2 inflammation gene signature, or a normalization of a subset of genes of a Type 2 inflammation gene signature. In some embodiments, the gene signature is the gene signature shown in FIG. 3, e.g., comprising the genes IL13RA1, FCER1A, CCL17, ARG1, IL4R, STAT6, CCR4, TSLP, DPP4, SIGLEC8, GATA1, PTGDR2, CCR3, CLC, HRH1, CCL24, ALOX15, CCL26, IL1RL1, HDC, TPSAB1, CMA1, IL25, IL4, GATA3, IL13, IL5, POSTN, CCL13, CCL18, IL33, CCL11, MUC5B, MUCSAC, PTGDS, and FCER2. In some embodiments, treatment with an IL-4R inhibitor results in a normalization of at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the genes of a Type 2 inflammation gene signature, e.g., a Type 2 inflammation gene signature as shown in FIG. 3. In some embodiments, normalization of a gene signature or a subset of genes of a gene signature is measured relative to baseline (e.g., a subject's gene signature (e.g., gene expression levels) prior to the onset of treatment), e.g., as measured at day 8, 15, 22, 25, 29, 36, 43, 50, 57, 64, 71, 85 or later following administration of the IL-4R inhibitor, or after 24 weeks of treatment with the IL-4R inhibitor.

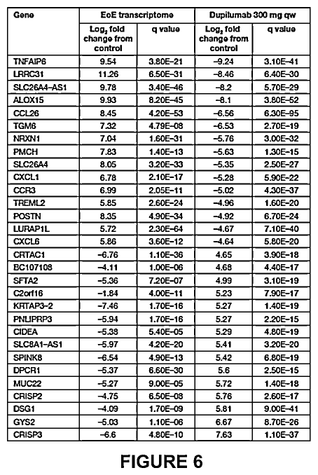

In some embodiments, treatment of a subject with an IL-4R inhibitor (e.g., an anti-IL-4R antibody) results in a normalization of the gene signature shown in FIG. 6, or a normalization of a subset of genes from the gene signature of FIG. 6, e.g., comprising the genes TNFAIP6, LRRC31, SLC26A4-AS1, ALOX15, CCL26, TGM6, NRXN1, PMCH, SLC26A4, CXCL1, CCR3, TREML2, POSTN, LURAP1L, CXCL6, CRTAC1, BC107108, SFTA2, C2orf16, KRTAP3-2, PLNIPRP3, CIDEA, SLC8A1-AS1, SPINK8, DPCR1, MUC22, CRISP2, DSG1, GYS2, and CRISP3. In some embodiments, treatment of a subject with an IL-4R inhibitor (e.g., an anti-IL-4R antibody) decreases, suppresses, or normalizes the expression of TARC (e.g., serum TARC), eotaxin-3 (e.g., plasma eotaxin-3), and/or IgE (e.g., serum total IgE).

### Combination Therapies

In some embodiments, the methods of the present disclosure comprise administering to the subject one or more additional therapeutic agents in combination with the IL-4R inhibitor. As used herein, the expression “in combination with” means that the additional therapeutic agents are administered before, after, or concurrent with the pharmaceutical composition comprising the IL-4R antagonist. The term “in combination with” also includes sequential or concomitant administration of IL-4R antagonist and a second therapeutic agent or therapy. In some embodiments, the second therapeutic agent or therapy is an IL-1β inhibitor, an IL-5 or IL-5R inhibitor (e.g., an anti-IL-5 or anti-IL-5R antibody such as benralizumab, mepolizumab, or reslizumab), an IL-9 inhibitor, an IL-13 inhibitor (e.g., an anti-IL-13 antibody such as tralokinumab, RPC4046, or QAX576), an IL-17 inhibitor, an IL-25 inhibitor, a TNFα inhibitor (e.g., an anti-TNFα antibody such as infliximab or adalimumab), an eotaxin-3 inhibitor, an IgE inhibitor (e.g., an anti-IgE antibody such as omalizumab), a TSLP inhibitor (e.g., an anti-TSLP antibody such as tezepelumab), a CRTH2 inhibitor, a Siglec-8 inhibitor, a prostaglandin D2 inhibitor, an integrin inhibitor (e.g., an integrin α4β7 inhibitor such as vedolizumab), an eotaxin inhibitor, an immunosuppressant, a topical corticosteroid, an oral corticosteroid, a systemic corticosteroid, an inhaled corticosteroid, a glucocorticoid, a PPI, a decongestant, an antihistamine, a non-steroidal anti-inflammatory drug (NSAID), esophagus dilation, allergen removal, or diet management. In some embodiments, the IL-4R inhibitor is used in combination with diet management. In some embodiments, the IL-4R inhibitor is used in combination with a corticosteroid (e.g., a swallowed topical corticosteroid).

In some embodiments, the IL-4R inhibitor is used in combination with a PPI, e.g., omeprazole, esomeprazole, lansoprazole, dexlansoprazole, rabeprazole, or pantoprazole. In some embodiments, the IL-4R inhibitor is used in combination with a high-dose PPI regimen. For example, in some embodiments, the IL-4R inhibitor is used in combination with: omeprazole at a dose of 40 mg QD or 20 mg BID, esomeprazole at a dose of 40 mg QD or 20 mg BID, lansoprazole at a dose of 60 mg QD or 30 mg BID, dexlansoprazole at a dose of 60 mg QD, rabeprazole at a dose of 40 mg QD or 20 mg BID, or pantoprazole at a dose of 80 mg QD or 40 mg BID.

In some embodiments, administration of the IL-4R inhibitor reduces dependence on or the need for using a concurrent therapy (e.g., a PPI, corticosteroid, or glucocorticoid). In some embodiments, administration of the IL-4R inhibitor in combination with the second therapy (e.g., PPI, corticosteroid, or glucocorticoid) reduces the amount of the second therapy (e.g., PPI, corticosteroid, or glucocorticoid) used by the patient by at least 20%, at least 30%, at least 40% or at least 50% as compared to the amount used by the subject before treatment with the IL-4R inhibitor.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial to Investigate the Efficacy and Safety of Dupilumab in Adult and Adolescent Patients with Eosinophilic Esophagitis

### Study Design and Objectives

This example describes a three-part, Phase 3, randomized clinical trial to investigate the efficacy and safety of dupilumab in adult and adolescent patients with eosinophilic esophagitis (EoE) (NCT03633617). Part A and Part B of the clinical trial are 24-week treatment, randomized, double-blind, placebo-controlled study phases and Part C is a 28 week, extended active treatment phase that will enroll patients from Part A and Part B. Dupilumab is a fully human anti-IL-4R antibody comprising a heavy chain comprising the amino acid sequence of SEQ ID NO:9 and a light chain comprising the amino acid sequence of SEQ ID NO:10; an HCVR/LCVR amino acid sequence pair comprising SEQ ID NOs:1/2; and heavy and light chain CDR sequences comprising SEQ ID NOs:3-8.

For Part A, the primary objectives of the study are to determine the treatment effect of dupilumab compared with placebo in adult and adolescent patients with EoE after 24 weeks of treatment, as assessed by histological and clinical measures, and to inform/confirm the final sample size determination for Part B. For Part B, the primary objective is to demonstrate the efficacy of dupilumab treatment compared with placebo in adult and adolescent patients with EoE after 24 weeks of treatment as assessed by histological and clinical measures. For Part C, the primary objective is to assess the safety and efficacy of dupilumab treatment in adult and adolescent patients with EoE after up to 52 weeks of treatment as assessed by histological and clinical measures. The secondary objectives of the study include: to evaluate the safety, tolerability, and immunogenicity of dupilumab treatment in adult and adolescent patients with EoE; to explore the relationship between dupilumab concentration and responses in adult and adolescent patients with EoE, using descriptive analyses; and to evaluate the effects of dupilumab on transcriptomic signatures associated with EoE and type 2 inflammation.

Part A consists of a screening period, randomization, and a treatment period as follows:
Screening period (up to 12 weeks): After obtaining informed consent, patients will initially be assessed for study eligibility at visit 1. Study participants are required to have a confirmed diagnosis of EoE, which may be established either by a prior historical biopsy or by biopsies performed during the screening period. All patients meeting clinical and laboratory eligibility criteria will undergo endoscopy with biopsies at visit 2 to establish a baseline reference measure. For patients without a historical biopsy, the visit 2 biopsies will serve as both confirmation of EoE diagnosis and the baseline reference measure.
Randomization: At the baseline visit (visit 3), patients who continue to meet eligibility criteria will enter the 24-week placebo-controlled, double-blind treatment period and will be randomized in a 1:1 ratio to dupilumab 300 mg once weekly (QW) or placebo administered subcutaneously (SC).
Placebo-controlled double-blind treatment period (24 weeks): The co-primary endpoints will be assessed at week 24, one week after the last dose of study drug during the double-blind treatment period to inform/confirm the final sample size determination for Part B. At the end of the double-blind treatment visit (week 24), eligible patients in Part A may enter a 28-week extended active treatment period (Part C). Patients not participating in Part C will enter a 12-week follow-up period. Patients enrolled in Part A will not be eligible to participate in Part B.
Follow-up period (12 weeks): All patients will be followed up for an additional 12 weeks after completing Part C or, if ineligible for Part C, immediately following Part A or B.

For Part B, enrollment is scheduled to begin immediately after the last patient is enrolled in Part A. The screening procedures for Part B are identical to those described above for Part A. For randomization, at the baseline visit (visit 3), patients who continue to meet eligibility criteria will enter the 24-week double-blind treatment period and will be randomized in a 1:1:1 ratio to dupilumab 300 mg QW, dupilumab 300 mg once every two weeks (Q2W), or placebo administered SC. The procedures for the placebo-controlled double-blind treatment period (24 weeks) are identical to those for Part A. At the end of the end of the double-blind treatment visit (week 24), eligible patients in Part B may enter a 28-week extended active treatment period (Part C). Patients not participating in Part C will enter a 12-week follow-up period.

For Part C, a 28-week extended active treatment period, at the end of the double-blind treatment visit (week 24), eligible patients in Part A and Part B may enter a 28-week extended active treatment period where all patients will receive active treatment with dupilumab but only patients in Part B will be blinded to treatment regimen in Part C. Patients from Part A who are randomized to placebo during the double-blind treatment period will receive dupilumab 300 mg QW during Part C. Patients from Part A who are randomized to dupilumab 300 mg QW during the double-blind treatment period will continue to receive dupilumab 300 mg QW during Part C. Patients from Part B who are randomized to placebo during the double-blind treatment period will be re-randomized in a 1:1 ratio to dupilumab 300 mg QW or dupilumab 300 mg Q2W. Patients randomized to dupilumab 300 mg Q2W will also receive matching placebo administered Q2W alternating with dupilumab doses so the injection frequency will match the other group for regimen-blinding purposes. All other patients will remain on the same dupilumab dose regimen to which they are randomized during the double-blind treatment period. All patients will be followed up for an additional 12 weeks after completing Part C or, if ineligible for Part C, immediately following Part A or B.

This study is being conducted in accordance with the provisions of the Declaration of Helsinki, the International Conference on Harmonization Good Clinical Practices guideline, and applicable regulatory requirements. The protocol was reviewed and approved by institutional review boards/ethics committees at all sites. Written informed consent was obtained from all adult patients. For adolescent patients, written informed consent or assent was obtained from the patient and written informed consent was obtained from the patient's parent(s) or legal guardian(s).

### Patient Population

This study enrolls adult males and females≥18 years of age and adolescent males and females≥12 to <18 years of age at the time of study entry with EoE.

Inclusion Criteria: A patient must meet the following criteria to be eligible for inclusion in the study: (1) male or female, ≥12 years of age; (2) a documented diagnosis of EoE by endoscopic biopsy prior to screening, as demonstrated by intraepithelial eosinophilic infiltration (peak cell count 15 eos/hpf) from at least one esophageal region and performed after at least 8 weeks of treatment with a high-dose PPI regimen. If the patient discontinued PPI therapy, the biopsy must have been performed within 2 weeks of the date of discontinuation. If a prior (historical) endoscopic biopsy meeting these criteria is not available (or no prior biopsy is available), patients who meet other clinical and laboratory eligibility criteria will undergo treatment with a high-dose PPI regimen for at least 8 weeks before their baseline endoscopy/biopsies. NOTE: If the patient is already using an acceptable high dose PPI regimen at the time of the screening visit, the baseline endoscopy may be scheduled at any point during the screening period after 8 weeks of treatment have been documented. (3) baseline endoscopic biopsies with a demonstration on central reading of intraepithelial eosinophilic infiltration (peak cell count≥15 eos/hpf) in at least 2 of the 3 biopsied esophageal regions (proximal, mid, or distal); (4) history (by patient report) of an average of at least 2 episodes of dysphagia (with intake of solids) per week in the 4 weeks prior to screening; (5) at least 4 episodes of dysphagia in the 2 weeks prior to baseline, documented via eDiary, at least 2 of which require liquids, coughing or gagging, vomiting, or medical attention to obtain relief; (6) completed at least 11 of 14 days of DSQ eDiary data entry in the 2 weeks prior to the baseline visit (visit 3); (7) baseline DSQ score≥10; (8) able to understand and complete study-related questionnaires; (9) willing and able to comply with clinic visits and study-related procedures; (10) provide informed consent signed by study patient or legally acceptable representative. For adolescents, parent or legal guardian must provide signed informed consent (patients must also provide separate informed assent to enroll in the study).

Exclusion Criteria: The following were exclusion criteria for Part A and Part B of the study: (1) body weight≤40 kg; (2) prior participation in a dupilumab clinical trial, or past or current treatment with dupilumab; (3) initiation or change of a food-elimination diet regimen or re-introduction of a previously eliminated food group in the 6 weeks prior to screening. Patients on a food-elimination diet must remain on the same diet throughout the study. (4) other causes of esophageal eosinophilia or the following conditions: hypereosinophilic syndrome and eosinophilic granulomatosis with polyangiitis (Churg-Strauss syndrome). NOTE: Patients with eosinophilic gastroenteritis are eligible, provided they meet other eligibility criteria. (5) active Helicobacter pylori infection; (6) history of achalasia, Crohn's disease, ulcerative colitis, celiac disease, and prior esophageal surgery; (7) any esophageal stricture unable to be passed with a standard, diagnostic, 9 to 10 mm upper endoscope or any critical esophageal stricture that requires dilation at screening; (8) history of bleeding disorders or esophageal varices that, in the opinion of the investigator, would put the patient at undue risk for significant complications from an endoscopy procedure; (9) treatment with swallowed topical corticosteroids within 8 weeks prior to baseline; (10) initiation, discontinuation, or change in the dosage regimen of the following medications within 8 weeks prior to the baseline endoscopy: proton pump inhibitors (except for patients who require a PPI trial prior to baseline endoscopy), leukotriene inhibitors, or nasal and/or inhaled corticosteroids. Patients on a stable dose of these medications for at least 8 weeks prior to the baseline endoscopy may be included in the study, but must not change the dose during the study. (11) initiation, discontinuation, or change in the dosage regimen of SC immunotherapy (SCIT). Patients on a stable dose of these medications for at least 1 year prior to visit 1 may be included in the study, but must not change the dose during the study. (12) treatment with sublingual immunotherapy (SLIT); (13) treatment with oral immunotherapy (OIT) within 6 months prior to visit 1; (14) the following treatments within 3 months prior to screening, or any condition that, in the opinion of the investigator, is likely to require such treatment(s) during the study: systemic immunosuppressant/immunomodulating drugs, including but not limited to systemic corticosteroids, omalizumab, cyclosporine, mycophenolate-mofetil, interferon-gamma [IFN-γ], Janus kinase inhibitors, azathioprine, and methotrexate (Note: one-time use of a corticosteroid as a part of the anesthetic preparation used during each endoscopy procedure is allowed); (15) treatment with an investigational drug within 2 months or within 5 half-lives (if known), whichever is longer, prior to visit 1; (16) planned or anticipated use of any prohibited medications and procedures during the study; (17) planned or anticipated major surgical procedure during the study; (18) treatment with a live (attenuated) vaccine within 4 weeks prior to the baseline visit; (19) active parasitic infection or suspected parasitic infection, unless clinical and (if necessary) laboratory assessments have ruled out active infection before randomization; (20) chronic or acute infection requiring treatment with systemic antibiotics, antivirals, or antifungals within 2 weeks before baseline visit; (21) known or suspected immunodeficiency disorder, including history of invasive opportunistic infections (e.g., tuberculosis [TB], non-tuberculous mycobacterial infections, histoplasmosis, listeriosis, coccidioidomycosis, pneumocystosis, aspergillosis) despite infection resolution, or otherwise recurrent infections of abnormal frequency, or prolonged infections suggesting an immune-compromised status, as judged by the investigator; (22) known history of human immunodeficiency virus (HIV) infection; (23) established diagnosis of hepatitis B viral infection at the time of screening or positive for hepatitis B surface antigen (HBsAg) at the time of screening. Patients who have gained immunity for hepatitis B virus infection after vaccination (patients who are HBsAg negative, hepatitis B surface antibody [HBsAb] positive, and hepatitis B core antibody [HBcAb] negative) are eligible for the study. Patients with positive HBcAb are eligible for the study only if hepatitis B virus DNA level is undetectable. (24) established diagnosis of hepatitis C viral (HCV) infection at the time of screening. Patients positive for hepatitis C Ab are eligible for the study only if HCV RNA is negative; (25) on current treatment for hepatic disease including but not limited to acute or chronic hepatitis, cirrhosis, or hepatic failure, or has evidence of liver disease as indicated by persistent (confirmed by repeated tests≥2 weeks apart) elevated transaminases (alanine aminotransferase [ALT] and/or aspartate aminotransferase [AST]) more than 3 times the upper limit of normal [ULN] during the screening period); (26) any of the following abnormal lab values at screening: platelets<100×10³/μL, neutrophils<1.5×10³/μL, or estimated glomerular filtration rate (eGFR)<30 mL/min/1.73 m²; (27) severe concomitant illness(es) that, in the investigator's judgment, would adversely affect the patient's participation in the study. Examples include but are not limited to short life expectancy, uncontrolled diabetes, cardiovascular conditions (e.g., NYHA Class III or IV cardiac failure), severe renal conditions (e.g., severe nephrotic syndrome), hepatobiliary conditions (e.g., Child-Pugh class B or C), neurological conditions (e.g., demyelinating diseases), active major autoimmune diseases (e.g., lupus, inflammatory bowel disease, rheumatoid arthritis, etc.), other severe endocrinologic, gastrointestinal, metabolic, pulmonary, or lymphatic diseases. (28) history of malignancy within 5 years prior to screening, except completely treated in situ carcinoma of the cervix and completely treated non-metastatic squamous or basal cell carcinoma of the skin; (29) history of alcohol or drug abuse within 6 months prior to screening; (30) any other medical or psychological condition including relevant laboratory abnormalities at screening that, in the opinion of the investigator, suggest a new and/or insufficiently understood disease, may present an unreasonable risk to the study patient as a result of his/her participation in this clinical trial, may make patient's participation unreliable, or may interfere with study assessments; (31) patient or his/her immediate family is a member of the investigational team; (32) pregnant or breastfeeding women, or women planning to become pregnant or breastfeed during the study; (33) women of childbearing potential who are unwilling to practice highly effective contraception prior to the initial dose/start of the first treatment, during the study, and for at least 12 weeks after the last dose. Postmenopausal women must be amenorrheic for at least 12 months in order not to be considered of childbearing potential. Pregnancy testing and contraception are not required for women with documented hysterectomy or tubal ligation. (34) known systemic hypersensitivity to dupilumab or the excipients of the drug product.

### Study Treatments

Dupilumab drug product was supplied for this study at a concentration of 150 mg/mL, with each 2.0 mL single-use prefilled glass syringe with snap-off cap delivering 300 mg of study drug (2.0 mL of a 150 mg/mL solution). Placebo matching dupilumab was prepared in the same formulation without the addition of protein (i.e., active substance, anti-IL-4Rα monoclonal Ab).

In the double-blind placebo-controlled Parts A and B, as well as Part C, all patients will receive once weekly (QW) subcutaneous (SC) injections. In Part A, all patients receive either 300 mg dupilumab or placebo QW. In Part B, patients will receive either 300 mg dupilumab QW, 300 mg dupilumab Q2W, or placebo QW. For the dupilumab 300 mg SC Q2W group, in order to maintain the blind, there will be an SC injection of placebo in between dupilumab doses so the injection frequency will match the other 2 groups (dupilumab QW and placebo). In the extended active treatment Part C, patients will receive dupilumab injections at the frequency (QW or Q2W with matching placebo alternating with dupilumab doses so the injection frequency will be identical for both groups for regimen-blinding purposes) per their treatment assignment. Subcutaneous injection sites of study drug should be alternated among the different quadrants of the abdomen (avoiding navel and waist areas), upper thighs, and upper arms so that the same site is not injected for 2 consecutive administrations.

Background treatment: Patients who undergo a trial of high-dose PPI therapy initiated prior to screening or during the screening period of Part A or Part B must remain on a dosage regimen listed below for the duration of the 52-week treatment period. A high-dose PPI regimen is defined as follows:
omeprazole 40 mg once a day (QD) or 20 mg twice a day (BID)
esomeprazole 40 mg QD or 20 mg BID
lansoprazole 60 mg QD or 30 mg BID
dexlansoprazole 60 mg QD
rabeprazole 40 mg QD or 20 mg BID
pantoprazole 80 mg QD or 40 mg BID

Patients who present at the initial screening visit with current use of PPIs must also remain on the same or similar approved dosage regimen for the duration of the 52-week treatment period. Patients may change to a different approved PPI medication during the study. PPI therapy is prohibited for all other patients.

Rescue treatments: If medically necessary (e.g., for treatment of intolerable EoE symptoms), rescue medications (systemic and/or swallowed topical corticosteroids) or emergency esophageal dilation are allowed for study patients. An endoscopy with biopsy will be performed prior to the initiation of rescue therapy. Patients who undergo an endoscopy with biopsy due to the initiation of rescue therapy will not undergo the scheduled end of treatment visit endoscopy/biopsy. Patients who receive rescue treatment during the double-blind period of the study will not be eligible for the extended active treatment period unless an endoscopy with biopsy is performed prior to the initiation of rescue treatment. However, if the endoscopy with biopsies cannot occur, rescue treatment should not be delayed, and these patients will be eligible for Part C. Part C treatment will be initiated per the schedule of events and only at an in-clinic visit. Patients receiving rescue therapy may continue to receive study drug. They will remain blinded and will be asked to return to the clinic for all remaining study visits for the double-blind treatment period and the follow-up period, and participate in all assessments for these visits according to the specified schedule of events. For the purpose of efficacy analyses, patients who receive rescue treatment during the study will be considered treatment failures.

### Outcomes Assessed

The co-primary endpoints for both Parts A and B of the study are: proportion of patients achieving peak esophageal intraepithelial eosinophil count of ≤6 eos/hpf at week 24; and absolute change in DSQ score from baseline to week 24.

The key secondary endpoints for both Parts A and B of the study are: absolute change in EoE-EREFS from baseline to week 24; percent change in peak esophageal intraepithelial eosinophil count (eos/hpf) from baseline to week 24; absolute change in EoE Grade Score from the EoE Histology Scoring System (EoEHSS) from baseline to week 24; and absolute change in EoE Stage Score from the EoEHSS from baseline to week 24.

Other secondary endpoints are: proportion of patients achieving peak esophageal intraepithelial eosinophil count of <15 eos/hpf at week 24; proportion of patients achieving peak esophageal intraepithelial eosinophil count of ≤1 eos/hpf at week 24; percent change in DSQ from baseline to week 24; normalized Enrichment Scores (NES) for the relative change from baseline to week 24 in the EoE diagnostic panel (EDP) transcriptome signature; NES for the relative change from baseline to week 24 in the type 2 inflammation transcriptome signature; absolute change from baseline to week 24 in severity and/or frequency of EoE symptoms other than dysphagia; proportion of patients who receive rescue medications or procedures during the 24 week placebo-controlled treatment period; and absolute change from baseline in esophageal distensibility plateau measured by functional lumen imaging, if collected, at week 24.

Procedures for assessing efficacy are described below and are also described in WO 2019/028367, incorporated by reference herein.

EoE-EREFS: The EoE esophageal characteristics will be analyzed based on the EoE-EREFS, a validated scoring system for inflammatory and remodeling features of disease using both overall scores and scores for each individual characteristic (Hirano, 2013). The proximal and distal esophageal regions are scored separately; the score for each region ranges from 0 to 9 and the overall score ranges from 0 to 18. The major esophageal features include: edema (absent, present); rings (absent, mild, moderate, severe); exudates (absent, mild, severe); furrows (absent, mild, severe); and stricture (absent, present). In addition to these major features, data for the following minor features are also captured by the physician performing the endoscopy procedure: crepe paper esophagus (mucosal fragility or laceration upon passage of diagnostic endoscope): absent, present; narrow caliber esophagus (reduced luminal diameter of the majority of the tubular esophagus): absent, present; and stricture diameter. Mucosal changes associated with gastroesophageal reflux disease also are recorded using the Los Angeles classification system for erosions (No Erosions or Grade A, B, C, or D).

Biopsies: Biopsies are obtained by endoscopy at the second screening visit (visit 2, day 21±7), week 24 and week 52 visits, and immediately prior to start of rescue medication or procedures during the double-blind treatment period. A total of 9 mucosal pinch biopsies are collected at each time point from 3 esophageal regions: 3 proximal, 3 mid, and 3 distal. Two samples from each region are used for the histology (needed for study inclusion criteria, as well as endpoint assessment) and other tissue analyses (may include but not limited, to immunohistochemistry [IHC], RNA scope (in situ hybridization), and RNA sequencing). The third sample from each region is processed for RNA analyses. In addition, biopsy specimens from the stomach and/or duodenum are obtained at visit 2 in all patients<18 years of age to rule out alternate etiologies of esophageal eosinophilia. Targeted, stomach and/or duodenal biopsies are obtained in adults only in the event of abnormal endoscopic findings (other than typical EoE findings) or clinical suspicion of alternate etiologies. Gastric biopsy samples should include 2 samples from the antrum and 2 samples from the body. Duodenal biopsy samples should include 2 bulb samples and 2 from another portion of the duodenum. Biopsy samples are assessed for peak eos per hpf and EoE Grade Scores and Stage Scores are assigned. EoE Grade and Stage Scores evaluate eight features: eosinophil density, basal zone hyperplasia, eosinophil abscesses, eosinophil surface layering, dilated intercellular spaces, surface epithelial alteration, dyskeratotic epithelial cells, and lamina propria fibrosis (absent/present).

EndoFLIP: The EndoFLIP device is a catheter-based procedure that measures the cross-sectional area at multiple sites along the esophagus with simultaneous intraluminal pressure recordings during volumetric distension of the esophagus. The analyses of cross-sectional area versus pressure relationships of the esophagus allow for determination of esophageal compliance as well as the distensibility plateau. The distensibility plateau has been shown to be significantly reduced in adult patients with EoE compared to healthy controls (Kwiatek, 2011). Moreover, the esophageal distensibility has been associated with outcomes of both food impaction and need for esophageal dilation (Nicodeme, 2013).

Dysphaqia Symptom Questionnaire (DSQ): The DSQ is a validated PRO that has been used in clinical studies to measure the frequency and intensity of dysphagia (Hudgens, et al., J Patient Rep Outcomes 2017, 1(1):3, doi:10.1186/s41687-017-0006-5). For patients who respond “No” to Question 1 (“Since you woke up this morning, did you eat solid food?”), a modification was made to the DSQ by asking a follow-up question to probe if patients avoided solid food due to their problems with swallowing solid food. This modified DSQ is completed by the patient daily using an eDiary from screening through end of study or ET visit. The DSQ uses a daily recall period and comprises 3 questions on the presence and severity of EoE dysphagia. All patients respond to questions 1 and 2 and are required to have eaten solid foods (‘Yes’ to question 1: “Since you woke up this morning, did you eat solid food?”) in order to proceed with the questionnaire. If a patient answers “No” to question 1, the remaining items on the DSQ are not scored. Patients who respond “No” to question 2 (“Since you woke up this morning, has food gone down slowly or been stuck in your throat?”) are given a score of zero, and do not go on to answer question 3 (the diary is recorded as completed for that day). Those who respond ‘Yes’ to questions 1 and 2 move on to question 3, which is scored on a 5-point scale that infers severity of dysphagia based on the patient's action to alleviate symptoms, ranging from no action to seeking medical attention. The DSQ scoring algorithm is therefore constructed from the responses to questions 2 and 3, to ensure that the final score is driven by the frequency and severity of dysphagia. To calculate the DSQ score, a minimum of 8 diary entries is required for each 14-day period to derive a standardized total score based on the cumulative scores through 14 days. DSQ scores can theoretically range from 0 to 84, with a lower score indicating less-frequent or less-severe dysphagia.

EoE Impact Questionnaire (EoE-IQ): The EoE-IQ is a disease-specific measure of health-related QOL in EoE patients developed by the sponsor. The EoE-IQ measures EoE impact on emotional, social, work and school, and sleep aspect of a patient on a scale of 1 to 5; higher scores indicate higher symptom burden. Concepts measured in the EoE-IQ (on a 5-point response option ranging from “not at all” to “extremely”) can include: “During the past 7 days, were you: bothered by symptoms of EoE; worried about trouble swallowing; worried about choking; embarrassed; worried about trouble swallowing while in a public place; difficulty taking part in social activities that involve eating food; impact of EoE on relationships with family; impact of EoE on relationships with friends; difficulty keeping up with things at work or school; missing work or school; sleep disruption.” The EoE-IQ is completed by patients using electronic questionnaire at specifically defined times during the study.

EoE Symptom Questionnaire (EoE-SQ): The EOE Symptom Questionnaire is a questionnaire measuring the frequency and severity of symptoms other than dysphagia and pain with swallowing on a scale of 5 to 25; higher scores indicate higher symptom burden. It is developed by the sponsor. Concepts measured in the EoE-SQ-Frequency (on a 5-point response option ranging from “never” to “more than once a day”) can include: “During the past 7 days, how often did you experience: chest pain; stomach pain; burning feeling in your chest (heartburn); food or liquid coming back up into your throat; throwing up.” The EoE Symptom Questionnaire is completed by patients using electronic questionnaire at specifically defined times during the study.

Patient Global Impression of Change (PGIC) of Dysphagia: The PGIC is a one-item questionnaire that asks patients to provide the overall self-assessment of change of difficulty swallowing food on a 7-point scale (very much better; moderately better; a little better; no change; a little worse; moderately worse; or very much worse). The PGIC is completed by patients using electronic questionnaire at specifically defined times during the study.

Total Nasal Symptom Score (TNSS): The Total Nasal Symptom Score (TNSS), measured on a 0-9 scale, is a composite symptom assessment of congestion, itching/sneezing, and rhinorrhea (each graded on a 0-3 scale, 3 being severe). The TNSS is administered only to patients with a documented history of allergic rhinitis and who fluently speak a language in which the questionnaire is presented (based on availability of translations in participating countries). The TNSS is completed by patients using electronic questionnaire at specifically defined times during the study.

Standardized Rhinoconjunctivitis Quality of Life Questionnaire for ages 12+ (RQLQ(S)+12): Standardized Rhinoconjunctivitis Quality of Life Questionnaire for ages 12+ [RQLQ(S)+12] is a self-administered questionnaire to measure health-related QOL in those 12 years of age and above, as a result of perennial or seasonal allergic rhinitis. There are 28 items on the RQLQ(S) in 7 domains: activity limitation, sleep problems, nasal symptoms, eye symptoms, non-nasal/eye symptoms, practical problems, and emotional function. The RQLQ(S)+12 responses are based on a 7-point Likert scale with responses ranging from 0 (not troubled) to 6 (extremely troubled). The overall RQLQ(S)+12 score is the mean of all 28 responses and the individual domain scores are the means of the items in those domains. Higher scores indicated more health-related QOL impairment (lower scores were better). A change of 0.5 point or more in total score is considered to be clinically meaningful. The RQLQ(S)+12 is completed by patients using electronic questionnaire at specifically defined times during the study.

Juniper Asthma Control Questionnaire (ACQ): The 5-question version of the Juniper ACQ (ACQ-5) is a validated questionnaire to evaluate asthma control. The ACQ-5 score is the mean of the scores of the 5 items and ranges from 0 (totally controlled) to 6 (severely uncontrolled). Scores less than 1.0 reflect adequately controlled asthma, and scores 1.0 or greater reflect inadequately controlled asthma. Higher score indicates lower asthma control. The recommended change of 0.50 is a reasonable threshold to define a meaningful individual-level change. The ACQ-5 is administered only to patients with a documented history of asthma and who fluently speak a language in which the questionnaire is presented (based on availability of validated translations in participating countries). The ACQ-5 is completed by patients using electronic questionnaire at specifically defined times during the study.

Patient-Oriented Eczema Measure (POEM): The POEM is a 7-item, validated questionnaire used in clinical practice and clinical trials to assess disease symptoms in children and adults with AD (Charman, 2004). The format is a response to 7 items (dryness, itching, flaking, cracking, sleep loss, bleeding, and weeping) using a 5-point scale, based on frequency of occurrence during the past week. The possible scores for each question were: 0 (no days), 1 (1 to 2 days), 2 (3 to 4 days), 3 (5 to 6 days), and 4 (every day), with a composite scoring system of 0 to 28; a higher score is indicative of more severe AD. The following POEM banding scores have been established: 0 to 2=clear or almost clear; 3 to 7=mild eczema; 8 to 16=moderate eczema; 17 to 24=severe eczema; and 25 to 28=very severe eczema. The POEM is administered only to patients with a documented history of AD and who fluently speak a language in which the questionnaire is presented (based on availability of validated translations in participating countries). The POEM is completed by patients using electronic questionnaire at specifically defined times during the study.

European Quality of Life 5-Dimensional Scale (EQ-5D): The European Quality of Life 5-dimension (EQ-5D) scale is a standardized questionnaire used to assess health status (Rabin, 2014). It consists of a descriptive system and the EQ visual analogue scale (EQ VAS). The EQ-5D-3L (3-level) descriptive system comprises of the following 5 dimensions: mobility, self-care, usual activities, pain/discomfort, and anxiety/depression. For each dimension, patients select one of 3 levels: no problems, some problems, and extreme problems. The EQ VAS records the patients' self-rated health on a vertical visual analogue scale where the endpoints are labelled ‘Best imaginable health state’ and ‘Worst imaginable health state’. The EQ-5D-3L is completed by patients using electronic questionnaire at specifically defined times during the study.

### Pharmacodynamic and Exploratory Biomarker Procedures

In this study, research assessments will be performed to explore EoE, how dupilumab may modify the underlying disease process in EoE, type 2 inflammation, and predictors of dupilumab safety and efficacy. Samples for eotaxin-3 (heparinized plasma); serum TARC, total IgE, and allergen-specific IgE and IgG4s are collected at specified time points. The biomarkers studied are believed to be relevant to the pathophysiology of EoE, response to treatment (i.e., assessment of type 2 inflammation) and baseline predictors of response and dupilumab mechanism of action.

### EoE Diagnostic Panel and Type 2 Inflammation Transcriptomics

The differential gene expression profiles of esophageal biopsies of EoE patients compared to healthy controls is the EoE disease transcriptome (Sherrill, et al., Genes Immun 2014, 15(6):361-369). This disease gene expression signature has been further refined to a smaller gene set to be used as an EoE diagnostic panel (EDP) (Dellon, et al., Clin Transl Gastroenterol 2017, 8(2):e74).

Normalized Enrichment Score (NES) reflects the degree to which the activity level of a set of transcripts is overrepresented at the extremes (top or bottom) of the entire ranked list of transcripts within a sample and is normalized by accounting for the number of transcripts in the set (Subramanian, 2005; Barbie, 2009). NES scores are calculated for each transcriptome signature for each sample by calculating the difference in expression levels between Baseline and Week 24 (TPM, transcripts per million) for all genes in the transcriptome, determining where a set of preselected transcripts falls on the distribution, calculating the enrichment score (the degree to which the difference in expression levels is in the extremes of the distribution) for each individual patient. For statistical comparison, once a NES is calculated for each individual, a Wilcoxon signed rank test is used to determine if there is a significant difference between the placebo and dupilumab groups.

### Results for Part A of Clinical Trial

### Baseline Characteristics

Baseline demographics and disease characteristics of patients enrolled in Part A of the study are summarized in Tables 1 and 2 below.

TABLE 1
Baseline Demographics

| | Placebo | | 300 mg QW | | Overall | |
|---|---|---|---|---|---|---|
| N (FAS) | 39 | | 42 | | 81 | |
| Age (years), | 28.8 | (12.53) | 33.9 | (15.53) | 31.5 | (14.31) |
| mean (SD) | | | | | | |
| ≥12 to <18 years, | 9 | (23.1) | 11 | (26.2) | 20 | (24.7) |
| n (%) | | | | | | |
| ≥18 years, n (%) | 30 | (76.9) | 31 | (73.8) | 61 | (75.3) |
| Gender (Male), | 21 | (53.8) | 28 | (66.7) | 49 | (60.5) |
| n (%) | | | | | | |
| Race, n (%) | | | | | | |
| White | 37 | (94.9) | 41 | (97.6) | 78 | (96.3) |
| Black or African | 1 | (2.6) | 1 | (2.4) | 2 | (2.5) |
| American | | | | | | |
| Other | 1 | (2.6) | 0 | | 1 | (1.2) |
| Weight (kg), | 74.5 | (15.45) | 80.9 | (24.81) | 77.8 | (20.95) |
| mean (SD) | | | | | | |
| BMI (kg/m²), | 25.8 | (5.79) | 26.3 | (6.79) | 26.1 | (6.30) |
| mean (SD) | | | | | | |

TABLE 2
Baseline Disease Characteristics

| | Placebo | | 300 mg QW | | Overall | |
|---|---|---|---|---|---|---|
| N (FAS) | 39 | | 42 | | 81 | |
| Duration of EoE (yr), mean (SD) | 4.77 | (4.546) | 5.23 | (4.177) | 5.01 | (4.337) |
| <5 years, n (%) | 26 | (66.7) | 24 | (57.1) | 50 | (61.7) |
| ≥5 years, n (%) | 13 | (33.3) | 18 | (42.9) | 31 | (38.3) |
| Prior swallowed topical steroid use, n (%) | 31 | (79.5) | 29 | (69) | 60 | (74.1) |
| Topical corticosteroid for EoE | | | | | | |
| effective, n (%) | | | | | | |
| Yes | 10 | (25.6) | 6 | (14.3) | 16 | (19.8) |
| No | 21 | (53.8) | 23 | (54.8) | 44 | (54.3) |
| History of prior esophageal dilations, n (%) | 17 | (43.6) | 18 | (42.9) | 35 | (43.2) |
| Number of prior esophageal dilation, mean | 2.0 | (1.41) | 1.9 | (1.16) | 2.0 | (1.27) |
| (SD) | | | | | | |
| Food elimination at screening, n (%) | 17 | (43.6) | 18 | (42.9) | 35 | (43.2) |
| Mean DSQ [0-84], mean (SD) | 35.1 | (12.11) | 32.2 | (12.66) | 33.6 | (12.41) |
| Mean Peak Eos count of 3 regions, mean | 96.5 | (54.69) | 82.6 | (41.02) | 89.3 | (48.29) |
| (SD) | | | | | | |
| Mean Eos count, mean (SD) | 70.30 | (40.93) | 58.71 | (33.808) | 64.29 | (37.623) |
| Blood peripheral Eos, mean (SD), Giga/L | 0.50 | (0.25) | 0.43 | (0.21) | 0.46 | (0.23) |
| Mean EoE Grade Score [0-3], mean (SD) | 1.324 | (0.4676) | 1.260 | (0.4088) | 1.291 | (0.4365) |
| Mean EoE Severity Score [0-3], mean (SD) | 1.376 | (0.3972) | 1.299 | (0.3334) | 1.336 | (0.3653) |
| Mean EREFS Score [0-18], mean (SD) | 6.0 | (2.38) | 6.5 | (3.20) | 6.3 | (2.83) |

Baseline demographics and disease characteristics were comparable across treatment groups, as shown in Tables 1 and 2. The mean DSQ for the population was around 34, indicating a substantial degree of baseline symptoms. A high percentage of subjects had a history of prior swallowed topical steroid use and prior esophageal dilations, as shown in Table 2. Baseline peak eosinophil counts were elevated. The majority of subjects also had at least one concurrent allergic condition (excluding EoE), as shown in Table 3.

TABLE 3
Concurrent Atopic/Allergic Conditions

| | Placebo | | 300 mg QW | | Overall | |
|---|---|---|---|---|---|---|
| N (SAS) | 39 | | 42 | | 81 | |
| # of patients with ≥1 | 36 | (92.3) | 33 | (78.6) | 69 | (85.2) |
| concurrent allergic condition | | | | | | |
| excluding EoE, n (%) | | | | | | |
| Allergic rhinitis, n (%) | 22 | (56.4) | 26 | (61.9) | 48 | (59.3) |
| Asthma, n (%) | 15 | (38.5) | 10 | (23.8) | 25 | (30.9) |
| Food allergy, n (%) | 17 | (43.6) | 19 | (45.2) | 36 | (44.4) |
| Atopic dermatitis, n (%) | 9 | (23.1) | 6 | (14.3) | 15 | (18.5) |
| Allergic conjunctivitis, n (%) | 7 | (17.9) | 6 | (14.3) | 13 | (16.0) |
| Baseline blood peripheral | 0.5 | (0.250) | 0.43 | (0.205) | 0.46 | (0.229) |
| eosinophils (Giga/L), | | | | | | |
| mean (SD) | | | | | | |
| Serum total IgE (IU/L), | 728.3 | (3176.19) | 356.4 | (673.28) | 535.5 | (2249.26) |
| mean (SD) | | | | | | |

### Efficacy

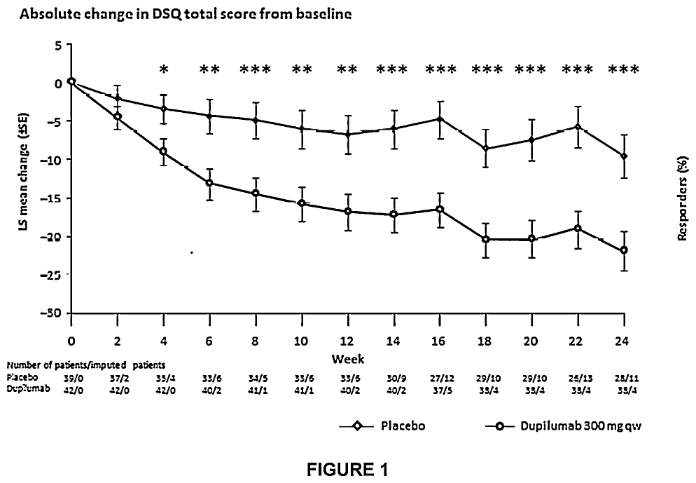

Treatment with dupilumab resulted in a statistically significant improvement in the co-primary endpoints (proportion of patients achieving peak esophageal intraepithelial eosinophil count of ≤6 eos/hpf at week 24, and absolute change in DSQ score from baseline to week 24) as shown in Table 7 below. The majority of patients receiving dupilumab at a dose of 300 mg QW (59.5%) achieved peak esophageal intraepithelial eosinophil count of 6 eos/hpf at week 24, versus 5.1% for placebo, consistent with histologic disease remission. Patients receiving dupilumab achieved a mean reduction in absolute DSQ score of 21.92, versus a reduction in absolute DSQ score of 9.60 for placebo. Absolute change in DSQ total score from baseline over time is shown in FIG. 1. Dupilumab significantly and rapidly reduced dysphagia severity in EoE patients. Greater improvements in DSQ score were seen for dupilumab- versus placebo-treated patients, reaching statistical significance from Week 4 onwards.

Treatment with dupilumab resulted in a statistically significant improvement in key secondary and other secondary endpoints, as shown in Table 7. Patients receiving dupilumab exhibited a −71.24% change in peak esophageal intraepithelial eosinophil count (eos/hpf) from baseline, versus a −2.98% change for placebo. A majority of patients receiving dupilumab (64.3%) achieved peak esophageal intraepithelial eosinophil count of <15 eos/hpf, versus only 7.7% of patients receiving placebo. A significant portion of patients receiving dupilumab achieved peak esophageal intraepithelial eosinophil count of ≤1 eos/hpf (21%, versus 0 patients on placebo). Dupilumab also significantly improved EoE histologic scores at Week 24; patients receiving dupilumab also exhibited a reduction in absolute change in Mean Grade Score and Mean Stage Score from the EOEHSS from baseline (Mean Grade Score: −0.761 for dupilumab vs. −0.001 for placebo; Mean Stage Score: −0.753 for dupilumab vs. −0.012 for placebo).

Dupilumab reduced endoscopic features of EoE at Week 24, as measured by EoE-EREFS which measures the severity of endoscopic findings; higher scores indicate greater severity. EREFS was assessed on endoscopy in the proximal and distal esophagus at screening and Week 24. Features assessed were edema (score range 0-1), rings (0-3), exudates (0-2), furrows (0-2), and strictures (0-1), with higher scores indicating the increasing severity. Total scores represent the sum of all features for both regions (0-18). Change from baseline in total scores, inflammation sub-score (sum of edema, exudates, and furrows), remodeling sub-score (sum of rings and stricture), and individual feature scores were analyzed at Week 24.

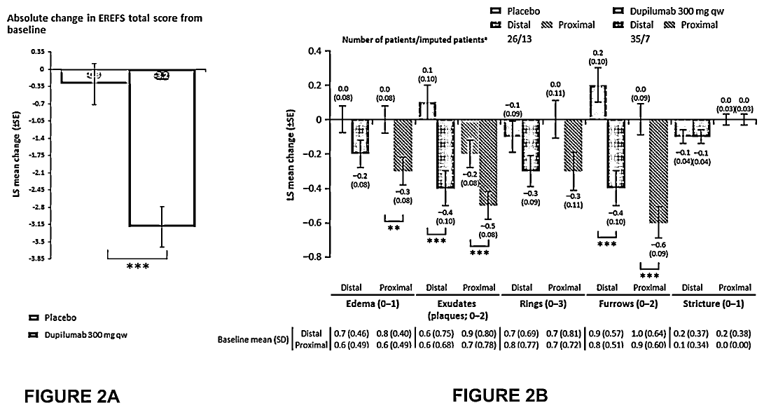

At baseline, mean [SD] total EREFS were similar for dupilumab- vs placebo-assigned patients (6.5 [3.20] vs 6.0 [2.38]) (Table 4). At Week 24, dupilumab- vs placebo-assigned patients had significantly greater change (improvement) in total EREFS and inflammation sub-score (both P<0.0001), and a trend towards improved remodeling sub-score. At Week 24, statistically nominally significant or numerical improvements were observed in proximal and distal edema (least square mean difference for dupilumab vs placebo of −0.3 and −0.1, respectively), rings (−0.2 and −0.2), exudates (−0.4 and −0.5), and furrows (−0.5 and −0.6), but not stricture (0 and 0) (FIG. 2B).

TABLE 4
Change from baseline to Week 24 in total EREFS and
inflammation and remodeling sub-scores
Total EoE-EREFS (range 0-18)

| | Placebo | Dupilumab 300 mg QW |
|---|---|---|
| | (N = 39) | (N = 42) |
| Baseline | | |
| Mean (SD) | 6.0 (2.38) | 6.5 (3.20) |
| Change from Baseline at Week 24 | | |
| No. of patients/imputed patientsª | 26/13 | 35/7 |
| LS mean (SE) | −0.3 (0.41) | −3.2 (0.41) |

| | |
|---|---|
| LS mean diff. vs placebo (95% CI)^{b} | −2.9 (−3.91 to −1.84) |
| P value vs placebo | P < 0.0001 |

EoE-EREFS sub-scores

| | Inflammation sub-score | | | |
|---|---|---|---|---|
| | (Edema + Exudates + Furrows; | | Remodeling sub-score | |
| | range 0-10) | | (Rings + Strictures; range 0-8) | |
| | | Dupilumab | | Dupilumab |
| | Placebo | 300 mg QW | Placebo | 300 mg QW |
| | (N = 39) | (N = 42) | (N = 39) | (N = 42) |
| Baseline | | | | |
| Mean (SD) | 4.2 (2.24) | 4.9 (2.69) | 1.8 (1.51) | 1.6 (1.56) |
| Change from Baseline at Week 24 | | | | |
| No. of patients/imputed patientsª | 26/13 | 35/7 | 26/13 | 35/7 |
| LS mean (SE) | 0.1 (0.35) | −2.3 (0.35) | −0.3 (0.17) | −0.7 (0.17) |
| LS mean diff. vs placebo (95% CI)^{b} | −2.4 (−3.30 to −1.54) | | −0.4 (−0.81 to 0.05) | |

| | | |
|---|---|---|
| Nominal P value vs placebo | <0.0001 | 0.0868 |

CI, confidence interval;
EoE-EREFS, Eosinophilic Esophagitis-Endoscopic Reference Score;
LS, least squares;
SD, standard deviation;
SE, standard error.
^{a}Five patients in the placebo group received rescue treatment; data after rescue treatment were set to missing and their Week 24 data were imputed. Other reasons for missing data include early discontinuation from study in Part A, Week 24 endoscopy was performed after patient took the first dose of Part C study drug, or Week 24 visit was delayed due to COVID-19 pandemic restriction.
^{b}The confidence interval with P value is based on treatment difference (dupilumab vs placebo) of the LS mean change using analysis of covariance model with baseline measurement as covariate and the treatment, age group (>=12 to <18 vs >=18) and PPI use at randomization strata as fixed factors.

Tables 4 and 7 and FIG. 2A show that patients treated with dupilumab exhibited an absolute change in EoE-EREFS total score from baseline of −3.2, versus −0.3 for placebo. FIG. 2B also shows decreases in EREFS component scores from baseline in patients treated with dupilumab versus placebo.

During the trial, patients completed the DSQ daily using an electronic diary. The DSQ score (range 0-84) was based on patients' response to questions on “food going down slowly or being stuck” and actions taken to “make the food go down or get relief.” The dysphagia-related pain score (range 0-56) was based on a follow-up question on “pain while swallowing”. Higher scores indicated greater severity. The proportion of patients achieving ≥30% and ≥50% reductions (commonly assessed PRO responder analysis thresholds) in the DSQ at Week 24 were analyzed using Cochran-Mantel-Haenszel test. Least squares (LS) mean change in dysphagia-related pain score from baseline to Week 24 was assessed by an analysis of covariance model. 76.2% of dupilumab vs 41.0% of placebo-treated patients achieved a ≥30% reduction in DSQ score (P<0.01), and 71.4% dupilumab vs 30.8% placebo-treated patients achieved a ≥50% reduction (P<0.001) (Table 5). Greater reductions in dysphagia-related pain score for dupilumab vs placebo were observed starting from Week 2 (LS mean difference: −2.3; 95% CI −4.4 to −0.1; P<0.05) and continued to decline to Week 24 (LS mean difference: −5.7; 95% CI −8.7 to −2.7; P<0.001). There was a −69.17% change in DSQ from baseline for patients treated with dupilumab, versus −31.68% change in DSQ for placebo-treated patients.

TABLE 5
DSQ responder analysis and dysphagia-related pain
score for dupilumab vs placebo in patients
enrolled in Part A of a 3-part EoE phase 3 study

| | |
|---|---|
| | Dupilumab |
| Placebo | 300 mg QW |
| (N = 39) | (N = 42) |

DSQ total score

| Baseline | | |
|---|---|---|
| Mean score (SD) | 35.1 (12.1) | 32.2 (12.7) |
| Week 24 | | |
| Proportion of patients with ≥30% reduction | 16 (41.0) | 32 (76.2) |
| in DSQ score from baseline, n (%) | | |
| Odds ratio vs placebo (95% CI) | 4.0 (1.6 to 9.8) | |
| Difference vs placebo, % (95% CI) | 34.7 (13.7 to 55.7) | |
| P-value | <0.01 | |
| Proportion of patients with ≥50% reduction | 12 (30.8) | 30 (71.4) |
| in DSQ score from baseline, n (%) | | |

| | |
|---|---|
| Odds ratio vs placebo (95% CI) | 4.9 (2.0 to 12.0) |
| Difference vs placebo, % (95% CI) | 40.1 (19.4 to 60.8) |
| P-value | <0.001 |

Dysphagia-related pain score

| | | |
|---|---|---|
| Baseline | | |
| Mean score (SD) | 13.3 (10.3) | 11.7 (9.5) |
| Change from baseline at Week 24 | | |
| Number of patients/imputed patients^{a} | 28/11 | 38/4 |
| LS mean (SE) | −4.4 (1.2) | −10.1 (1.1) |

| | |
|---|---|
| LS mean difference vs placebo (95% CI) | −5.7 (−8.7 to −2.7) |
| P-value | <0.001 |

CI, confidence interval;
DSQ, Dysphagia Symptom Questionnaire;
LS, least squares;
qw, weekly;
SD, standard deviation;
SE, standard error.
^{a}n = number of observed/imputed patients. Five patients in the placebo group received rescue treatment; data after rescue treatment were set to missing and their Week 24 data were imputed by multiple imputations.

The Eosinophilic Esophagitis Histologic Scoring System (EoE-HSS) evaluates the severity (grade) and extent (stage) of histological features, which correlate with clinical symptoms, in EoE esophageal biopsies. Biopsies were taken from proximal, mid, and distal esophageal regions, and the severity and extent of abnormality of 8 features (basal zone hyperplasia [BZH], eosinophil inflammation [EI], eosinophil abscess [EA], eosinophil surface layering [ESL], dilated intercellular spaces [DIS], surface epithelial alteration [SEA], dyskeratotic epithelial cells [DEC], and lamina propria fibrosis [LPF]) were scored by a central pathologist (0 normal-3 maximum change). Mean EoE-HSS grade or stage scores were calculated from the sum of assigned scores divided by the maximum possible score for each esophageal region; the 3 scores from each region were summed to make the total score. Absolute change from baseline to Week 24 in total and individual feature scores for grade and stage were analyzed.

Baseline characteristics were similar for dupilumab vs placebo groups, including mean EoE-HSS grade (1.26 vs 1.32) and stage (1.30 vs 1.38) total scores. At Week 24, dupilumab vs placebo had improved mean EoE-HSS total grade and stage scores (both P<0.001); significant improvements were observed in most histological features including BZH, EI, EA, ESL, DIS, SEA grade and BZH, EI, EA, ESL, SEA stage scores; all P<0.05 (Table 6). Numerical improvements were seen for dupilumab vs placebo in DEC and LPF grade and stage scores. LPF was often missing in biopsies resulting in reduced sample size. These data document the beneficial effect of dupilumab on non-eosinophil components of esophageal epithelium, including BZH and DIS.

TABLE 6
Change from baseline to Week 24 in EoE-HSS grade and
stage total and individual feature scores

| EoE-HSS total grade | | EoE-HSS total stage | |
|---|---|---|---|
| score (range 0-3) | | score (range 0-3) | |
| | Dupilumab | | Dupilumab |
| Placebo | 300 mg QW | Placebo | 300 mg QW |
| (N = 39) | (N = 42) | (N = 39) | (N = 42) |

Total score

| Baseline | | | | | | | | |
|---|---|---|---|---|---|---|---|---|
| Mean score (SD) | 1.32 | (0.47) | 1.26 | (0.41) | 1.38 | (0.40) | 1.30 | (0.33) |
| Change from baseline at | | | | | | | | |
| Week 24 | | | | | | | | |
| No. pts/imputed pts^{b} | 26/13 | | 35/7 | | 26/13 | | 35/7 | |
| LS mean (SE) | −0.001 | (0.06) | −0.76 | (0.06) | −0.01 | (0.06) | −0.75 | (0.06) |
| LS mean diff. vs placebo | −0.76 | | (−0.91 to −0.61) | | −0.74 | | (−0.88 to −0.60) | |
| (95% CI)^{c} | | | | | | | | |

| | | |
|---|---|---|
| P value vs placebo | <0.001 | <0.001 |

Basal zone hyperplasia (BZH)

| Baseline | | | | | | | | |
|---|---|---|---|---|---|---|---|---|
| Mean score (SD) | 2.59 | (0.79) | | 2.60 (0.59) | 2.80 | (0.61) | 2.95 | (0.22) |
| Change from baseline at | | | | | | | | |
| Week 24 | | | | | | | | |
| No. pts/imputed pts^{b} | 26/13 | | | 35/7 | 26/13 | | 35/7 | |
| LS mean (SE) | −0.01 | (0.14) | −1.86 | (0.14) | 0.02 | (0.18) | −1.90 | (0.18) |
| LS mean diff. vs placebo | | −1.85 | (−2.20 to −1.49) | | −1.91 | | (−2.37 to −1.46) | |
| (95% CI)^{c} | | | | | | | | |

| | | |
|---|---|---|
| P value vs placebo | <0.0001 | <0.0001 |

Eosinophil inflammation (EI)

| Baseline | | | | | | | | |
|---|---|---|---|---|---|---|---|---|
| Mean score (SD) | 2.67 | (0.48) | 2.67 | (0.48) | 2.72 | (0.60) | 2.57 | (0.59) |
| Change from baseline at | | | | | | | | |
| Week 24 | | | | | | | | |
| No. pts/imputed pts^{b} | 26/13 | | 35/7 | | 26/13 | | 35/7 | |
| LS mean (SE) | −0.05 | (0.11) | −1.21 | (0.11) | −0.02 | (0.15) | −1.90 | (0.15) |
| LS mean diff. vs placebo | −1.15 | | (−1.44 to −0.87) | | −1.88 | | (−2.27 to −1.49) | |
| (95% CI)^{c} | | | | | | | | |

| | | |
|---|---|---|
| P value vs placebo | <0.0001 | <0.0001 |

Eosinophil abscesses (EA)

| Baseline | | | | | | | | |
|---|---|---|---|---|---|---|---|---|
| Mean score (SD) | 0.85 | (0.90) | 0.83 | (0.85) | 0.62 | (0.59) | 0.60 | (0.50) |
| Change from baseline at | | | | | | | | |
| Week 24 | | | | | | | | |
| No. pts/imputed pts^{b} | 26/13 | | 35/7 | | 26/13 | | 35/7 | |
| LS mean (SE) | 0.16 | (0.09) | −0.63 | (0.09) | 0.25 | (0.07) | −0.42 | (0.07) |
| LS mean diff. vs placebo | −0.79 | | (−1.01 to −0.57) | | −0.67 | | (−0.85 to −0.49) | |
| (95% CI)^{c} | | | | | | | | |

| | | |
|---|---|---|
| P value vs placebo | <0.0001 | <0.0001 |

Eosinophil surface layering (ESL)

| Baseline | | | | | | | | |
|---|---|---|---|---|---|---|---|---|
| Mean score (SD) | 1.74 | (1.07) | 1.91 | (1.08) | 0.90 | (0.60) | 0.83 | (0.44) |
| Change from baseline at | | | | | | | | |
| Week 24 | | | | | | | | |
| No. pts/imputed pts^{b} | 26/13 | | 35/7 | | 26/13 | | 35/7 | |
| LS mean (SE) | 0.20 | (0.14) | −1.53 | (0.14) | 0.01 | (0.07) | −0.69 | (0.07) |
| LS mean diff. vs placebo | −1.74 | | (−2.09 to −1.38) | | −0.70 | | (−0.88 to −0.53) | |
| (95% CI)^{c} | | | | | | | | |

| | | |
|---|---|---|
| P value vs placebo | <0.0001 | <0.0001 |

Dilated intercellular spaces (DIC)

| Baseline | | | | | | | | |
|---|---|---|---|---|---|---|---|---|
| Mean score (SD) | 2.18 | (0.39) | 2.10 | (0.30) | 2.97 | (0.16) | 3.00 | (0.00) |
| Change from baseline at | | | | | | | | |
| Week 24 | | | | | | | | |
| No. pts/imputed ptsb | 26/13 | | 35/7 | | 26/13 | | 35/7 | |
| LS mean (SE) | 0.02 | (0.07) | −0.17 | (0.07) | −0.02 | (0.04) | −0.09 | (0.04) |
| LS mean diff. vs placebo | −0.18 | | (−0.36 to −0.01) | | −0.07 | | (−0.18 to 0.04) | |
| (95% CI)^{c} | | | | | | | | |

| | | |
|---|---|---|
| P value vs placebo | 0.0397 | 0.2092 |

Surface epithelial alteration (SEA)

| Baseline | | | | | | | | |
|---|---|---|---|---|---|---|---|---|
| Mean score (SD) | 1.13 | (1.17) | 1.14 | (1.12) | 0.95 | (1.07) | 0.95 | (1.06) |
| Change from baseline at | | | | | | | | |
| Week 24 | | | | | | | | |
| No. pts/imputed ptsb | 26/13 | | 35/7 | | 26/13 | | 35/7 | |
| LS mean (SE) | 0.33 | (0.15) | −0.71 | (0.14) | 0.27 | (0.14) | −0.60 | (0.14) |
| LS mean diff. vs placebo | −1.04 | | (−1.41 to −0.68) | | −0.87 | | (−1.22 to −0.52) | |
| (95% CI)^{c} | | | | | | | | |

| | | |
|---|---|---|
| P value vs placebo | <0.0001 | <0.0001 |

Dyskeratotic epithelial cells (DEC)

| Baseline | | | | | | | | |
|---|---|---|---|---|---|---|---|---|
| Mean score (SD) | 0.51 | (0.60) | 0.33 | (0.61) | 0.46 | (0.51) | 0.26 | (0.45) |
| Change from baseline at | | | | | | | | |
| Week 24 | | | | | | | | |
| No. pts/imputed ptsb | 26/13 | | 35/7 | | 26/13 | | 35/7 | |
| LS mean (SE) | −0.07 | (0.08) | −0.25 | (0.08) | −0.01 | (0.08) | −0.18 | (0.08) |
| LS mean diff. vs placebo | −0.19 | | (−0.39 to 0.01) | | −0.16 | | (−0.36 to 0.04) | |
| (95% CI)^{c} | | | | | | | | |

| | | |
|---|---|---|
| P value vs placebo | 0.0644 | 0.1067 |

Lamina propria fibrosis (LPF)

| Baseline | | | | | | | | |
|---|---|---|---|---|---|---|---|---|
| Mean score (SD) | 1.70 | (1.11) | 1.09 | (0.90) | 2.44 | (1.16) | 2.17 | (1.34) |
| Change from baseline at | | | | | | | | |
| Week 24 | | | | | | | | |
| No. pts/imputed ptsb | 9/14 | | 7/16 | | 9/14 | | 7/16 | |
| LS mean (SE) | −0.10 | (0.20) | −0.37 | (0.21) | −0.19 | (0.27) | −0.55 | (0.28) |
| LS mean diff. vs placebo | −0.27 | | (−0.79 to 0.25) | | −0.36 | | (−1.07 to 0.34) | |
| (95% CI)^{c} | | | | | | | | |

| | | |
|---|---|---|
| P value vs placebo | 0.3055 | 0.3136 |

CI, confidence interval;
EOE-HSS, Eosinophilic Esophagitis Histology Scoring System,
LS, least squares;
SD, standard deviation;
SE, standard error.
^{a}The mean grade or mean stage score from EoE-HSS for each biopsy is the sum of the assigned scores for each feature evaluated divided by the maximum possible score for that region (i.e., 24 unless a feature was not evaluated in which case the maximum possible score was reduced by 3).
^{b}Five pts in the placebo group received rescue treatment; data after rescue treatment were set to missing and their Week 24 data were imputed. Other reasons for missing data include early discontinuation from study in Part A, Week 24 endoscopy was performed after pt took the first dose of Part C study drug, or Week 24 visit was delayed due to COVID-19 pandemic restriction.
^{c}The confidence interval (CI) with p-value is based on treatment difference (Dupilumab group vs. Placebo) of the LS mean change using ANCOVA model with baseline measurement as covariate and the treatment, age group [>=12 to <18 vs >=18] and PPI use at randomization (Yes vs. No) strata as fixed factors.

A total of 5 placebo patients received rescue therapy: swallowed topical steroids (3), systemic steroids (1), or dilation (1). No patients treated with dupilumab utilized rescue therapy.

TABLE 7
Primary and Secondary Endpoints

| | | EE-1664 Part A FAS | | | | | | |
|---|---|---|---|---|---|---|---|---|
| | | | | | | Treatment | | |
| | | | | 300 mg | | Difference | | |
| | Endpoint | Placebo | | QW | | (95% CI) | | P value |
| | N (SAS) | 39 | | 42 | | | | |
| Co- | Proportion of patients achieving | 2 | (5.1) | 25 | (59.5) | 55.3 | (39.58, 71.04) | <0.0001 |
| Primary | peak esophageal intraepithelial | | | | | | | |
| | eosinophil count of | | | | | | | |
| | ≤6 eos/hpf, n (%) | | | | | | | |
| | Absolute change in DSQ | −9.60 | (2.785) | −21.92 | (2.526) | −12.32 | (−19.107, −5.537) | 0.0004 |
| | score (0-84). | | | | | | | |
| | LS mean difference (95% CI) | | | | | | | |
| Key | Percent change in peak | −2.98 | (7.596) | −71.24 | (6.948) | −68.26 | (−86.896, −49.615) | <0.0001 |
| Secondary | esophageal intraepithelial | | | | | | | |
| | eosinophil count (eos/hpf) | | | | | | | |
| | from baseline | | | | | | | |
| | Absolute change in Mean Grade | −0.001 | (0.0588) | −0.761 | (0.0573) | −0.758 | (−0.9061, −0.6127) | <0.0001 |
| | Score from the Histology Scoring | | | | | | | |
| | System (EoEHSS) from baseline | | | | | | | |
| | Absolute change in Mean | −0.012 | (0.0571) | −0.753 | (0.0557) | −0.741 | (−0.9061, −0.6127) | <0.0001 |
| | Stage Score from the | | | | | | | |
| | EoEHSS from baseline | | | | | | | |
| | Absolute change in EoE- | −0.3 | (0.41) | −3.2 | (0.41) | −2.9 | (−3.91, −1.84) | <0.001 |
| | Endoscopic Reference Score | | | | | | | |
| | (EoE-EREFS) from baseline | | | | | | | |
| Other | Proportion of patients | 3 | (7.7) | 27 | (64.3) | 57.5 | (41.69, 73.33) | <0.0001 |
| Secondary | achieving peak esophageal | | | | | | | |
| | intraepithelial eosinophil | | | | | | | |
| | count of <15 eos/hpf, n (%) | | | | | | | |
| | Proportion of patients achieving | 0 | | 9 | (21.4) | | | <0.001 |
| | peak esophageal intraepithelial | | | | | | | |
| | eosinophil count of | | | | | | | |
| | ≤1 eos/hpf, n (%) | | | | | | | |

| | | | | | |
|---|---|---|---|---|---|
| Percent change in DSQ | −31.68 | −69.17 | −37.48 | (−57.222, −17.745) | 0.0002 |
| from baseline | | | | | |

### Safety

Safety data for the trial is shown in Table 8, below. No new or unexpected side effects were observed in the trial, and no related SAEs were observed. Dupilumab was well tolerated, and the majority of AEs were mild in intensity.

TABLE 8
Safety Assessment

| | Part A | |
|---|---|---|
| | Placebo | 300 mg QW |
| % of Patients | (N = 39) | (N = 42) |
| Deaths | 0 | 0 |
| TEAEs, n (%) | 32 (82.1) | 36 (85.7) |
| SAEs, n (%) | 0 | 2 (4.8) |
| AEs leading to treatment discontinuation, n (%) | 0 | 1 (2.4) |
| TEAE of Special Interest (Adjudicated) | 0 | 0 |
| Conjunctivitis (Broad CMQ) | 1 (2.6) | 2 (4.8) |
| Infections and Infestations (SOC) | 10 (25.6) | 15 (35.7) |
| Upper Respiratory Infections (HLT) | 6 (15.4) | 11 (26.2) |

SAEs:
61-year-old female with abdominal pain; colon polyp removal 3 days prior; unrelated to study medication
47-year-old female with worsening endometrial polyp resulting in sub-total abdominal hysterectomy

### Gene Expression Profiles

The impact of dupilumab treatment on the differential gene expression profile was evaluated by RNA sequencing using tissue acquired via esophageal biopsies from the patients in this study as compared to tissue profiles from pre-treatment baseline. It is known that gene expression dysregulated in disease (the EoE disease transcriptome) includes not only those genes related to eosinophils and type 2 inflammation, but also those related to epithelial proliferation, barrier function, remodeling and fibrosis. Utilization of scores derived from transcriptome signatures provides a mechanism to quantitate the overall dysregulation of disease and target inflammatory pathway. Moreover, the transcriptomes give quantitative, molecular phenotypes of disease that include parameters associated not only with inflammation, but also epithelial barrier, remodeling and fibrosis that may not be readily measured by other methodologies.

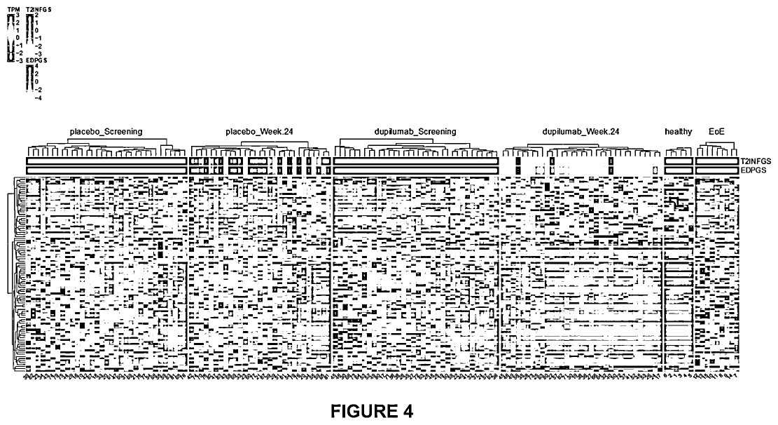

In Part A, treatment with dupilumab, but not placebo, suppressed both the type 2 inflammation and EoE diagnostic panel (EDP) normalized enrichment scores (NES). See, FIGS. 3 and 4. The type 2 inflammation transcriptome is a Regeneron-curated gene list of genes associated with type 2 inflammation as shown in FIG. 3. The EDP is a published 96-gene panel of genes differentially expressed in esophageal pinch biopsies from EoE patients vs controls (Wen, et al., Gastroenterology 2013; 145(6):1289-1299). As shown in Table 9 below, dupilumab decreased the EoE disease signature in esophageal biopsies by −2.66 (compared with −0.160 with placebo) to a phenotype more similar to normal esophageal tissue, and decreased the type 2 inflammation signature by −1.97 from baseline (compared with −0.32 with placebo).

TABLE 9
Type 2 and EoE Diagnostic Panel Gene Expression Signatures

| | | | |
|---|---|---|---|
| Placebo | Dupilumab | | |
| Relative | Relative | | |
| change | change | Treatment | |
| from baseline | from baseline | Difference | |
| NES (n) | NES (n) | (95% CI) | P value |

Type 2 inflammation NES

| | | | | | |
|---|---|---|---|---|---|
| Adults + | −0.320 (n = 29) | −1.970 (n = 31) | −1.590* | (−1.7400, −1.2700) | <0.0001 |
| adolescents | | | | | |
| Adolescents | −0.170 (n = 5) | −1.980 (n = 6) | −1.810* | (−3.1300, −0.8500) | 0.0081 |

EoE Diagnostic Panel (EDP) NES

| | | | | | |
|---|---|---|---|---|---|
| Adults + | −0.160 (n = 29) | −2.660 (n = 31) | −2.250^{#} | (−2.7200, −1.7300) | <0.0001 |
| adolescents | | | | | |
| Adolescents | −0.050 (n = 5) | −2.705 (n = 6) | −2.655* | (−4.0800, −1.1500) | 0.0081 |

*Median difference
^{#}Mean difference

### Other Endpoints

HRQoL was assessed by the 11-item EoE Impact Questionnaire (EoE-IQ), measuring emotional, social, productivity, and sleep-related impacts of EoE (score range: 1-5). Symptom burden was assessed by the 5-item EoE Symptom Questionnaire (EoE-SQ-Frequency), measuring frequency of EoE symptoms other than dysphagia/swallowing pain, including chest pain, stomach pain, heartburn, regurgitation, and vomiting (score range: 5-25). Higher EoE-IQ/EoE-SQ-Frequency scores indicate greater impact on HRQoL/symptom burden. Proportion of patients reporting dysphagia improvement on the Patient Global Impression of Change (PGIC) was evaluated. The PGIC asked patients to rate their overall change in difficulty swallowing food since they started taking the study treatment on a 7-point scale, ranging from “Very much better” to “Very much worse.”

### Results

At baseline, mean EoE-IQ was 2.0/2.4 and mean EoE-SQ-Frequency 10.1/11.5 in dupilumab/placebo groups, respectively. At Week 24, LS mean change from baseline difference for dupilumab versus placebo was −0.4 (95% CI: −0.6, −0.1; nominal P=0.008) for EoE-IQ and −1.7 (−2.9, −0.5; nominal P=0.005) for EoE-SQ-Frequency. At Week 24, 40.5% versus 7.7% (nominal P<0.001) of dupilumab versus placebo patients reported dysphagia as “very much better” compared with baseline on the PGIC; 26.2% versus 10.3% (nominal P=0.074) reported “moderately better”.

### Biomarker Analysis

The effect of dupilumab on circulating biomarkers of type 2 inflammation over the 24-week treatment period was analyzed. Median and median change from baseline in biomarkers, serum thymus and activation-regulated chemokine (TARC), plasma eotaxin-3, and serum total immunoglobulin E (IgE), were assessed at Weeks 4, 12 and 24.

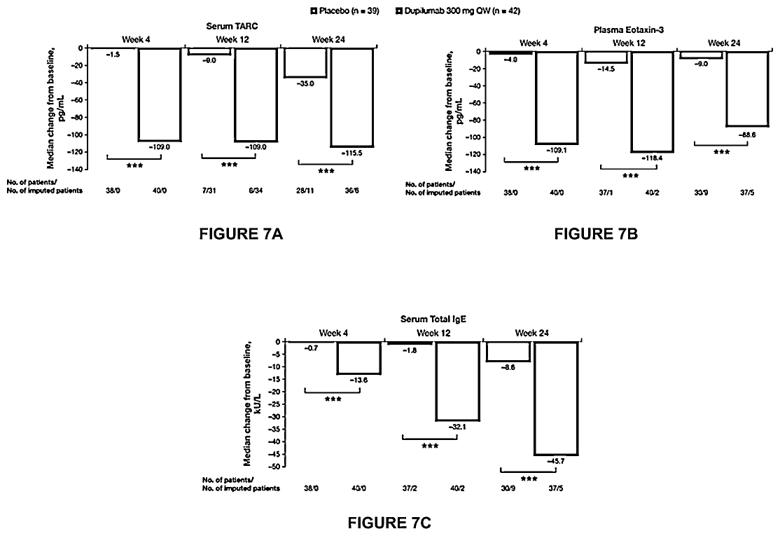

Baseline levels of TARC, eotaxin-3, and total IgE were similar across treatment groups (median [Q1-Q3] for dupilumab vs placebo in TARC: 322.0 pg/mL [232.0-430.0] vs 293.0 pg/mL [226.0-418.0]; eotaxin-3: 217.5 pg/mL [139.0-330.0] vs 217.0 pg/mL [163.0-448.0]; total IgE: 110.0 kU/L[51.1-463.0] vs 100.0 kU/L [46.7-294.0]). In dupilumab- vs placebo-treated patients, TARC and eotaxin-3 decreased rapidly from baseline with effects sustained over 24 weeks, whereas total IgE decreased more gradually (FIG. 7A-7C). At Week 24, median (Q1-Q3) for dupilumab vs placebo in TARC was 196.5 pg/mL (134.0-277.0) vs 319.0 pg/mL (191.0-381.0), eotaxin-3 was 110.0 pg/mL (82.3-133.0) vs 203.0 pg/mL (164.0-358.0), and total IgE was 59.8 kU/L (21.7-161.0) vs 106.0 kU/L (42.7-228.0) (all P<0.0001 for dupilumab vs placebo in median change from baseline). Median changes from baseline for dupilumab vs placebo at Weeks 4, 12, and 24, respectively, were as follows: TARC, −109.0 pg/mL vs −1.5 pg/mL, −109.0 pg/mL vs −9.0 pg/mL, −115.5 pg/mL vs −35.0 pg/mL; eotaxin-3, −109.1 pg/mL vs −4.0 pg/mL, −118.4 pg/mL vs −14.5 pg/mL, −88.6 pg/mL vs −9.0 pg/mL; total IgE, −13.6 kU/L vs −0.7 kU/L, −32.1 kU/L vs −1.8 kU/L, −45.7 kU/L vs −8.6 kU/L (all P<0.0001).

In summary, over the 24-week treatment period, dupilumab treatment led to rapid and sustained suppression of serum TARC and plasma eotaxin-3, and gradual suppression of serum total IgE in adolescents and adults with EoE. Consistent with prior findings in EoE and other type 2 inflammatory diseases, these results demonstrate IL-4/IL-13-dependent regulation of type 2 inflammation.

### Conclusion

The Part A data for this Phase 3 clinical trial demonstrates that weekly administration of dupilumab is effective at improving dysphagia and histological and endoscopic measures for the treatment of EoE. The trial met both of its co-primary endpoints (absolute change in DSQ and the proportion of patients achieving peak esophageal intraepithelial eosinophil counts of ≤6 eos/hpf at Week 24), as well as all key secondary endpoints. Notably, this is the first time a Phase 3 trial with a biologic has reported improvement in patients' ability to swallow food, as reported by the validated DSQ. 60% of patients treated with dupilumab reduced their esophageal eosinophil count to a normal range, compared with 5% of placebo-treated patients. Dupilumab also reduced abnormal endoscopic findings compared with placebo, as measured by EREFS. Furthermore, transcriptional profiling showed that dupilumab normalized the expression of genes associated with EoE, including those related to eosinophils and type 2 inflammation, epithelial proliferation, barrier function, remodeling and fibrosis, indicating a molecular reversal of disease beyond a reduction in eosinophilic inflammation.

### Example 2: Transcriptome Analysis of Patients with Eosinophilic Esophagitis Treated with Dupilumab

This example describes the results of a transcriptome analysis for adult patients (aged 18-65 years) enrolled in a Phase 2 clinical trial (NCT02379052). Transcriptome results were available for 19 of the 24 placebo patients (79%) and 22 of the 23 dupilumab patients (96%) enrolled in the phase 2 trial; biopsy specimens were not available from 6 patients, who were excluded from the analysis.

### Methods

Patients completed a 35-day screening period, followed by randomization 1:1 to receive subcutaneous injections of dupilumab 300 mg (loading dose of 600 mg on Day 1) once-weekly (QW) or matched placebo for 12 weeks, and a 16-week follow-up period. Pinch biopsies for RNA analysis were collected and frozen in RNALater from the proximal, mid, and distal esophagus during the screening and Week 12 endoscopy procedures. After RNA extraction, strand-specific RNA-seq libraries were prepared using KAPA stranded mRNA-Seq Kit (KAPA Biosystems, Roche Sequencing and Life Sciences, MA, USA). After amplification, sequencing was performed on Illumina HiSeq®2000 (Illumina Inc., CA, USA) by multiplexed single-read run (80 bp, 40M reads). Reads were mapped to the human genome (National Center for Biotechnology Information GRCh37) using Array Studio software (OmicSoft, NC, USA). Differentially expressed genes were identified using the DESeq2 package.

Using a gene set enrichment analysis tool that takes both positive and negative gene sets into consideration (www.mathworks.com/matlabcentral/fileexchange/33599-gsea2), the top 50 most upregulated and top 50 most downregulated genes in EoE were used to generate a normalized enrichment score (NES). Gene expression profiles were first transformed into z-scores, and single-sample NES was computed using the ranked z-scores in each sample to represent the sample's overall disease signature score, denoted as EoE-NES.

Unbiased global transcriptome analysis was performed using Gene Set Enrichment Analysis (GSEA) with GO biological process gene sets from the Molecular Signatures Database (MSigDB, c5.bp.v7.0). Gene sets with size>100 were prefiltered to ensure biological process specificity, and top GO terms were selected if FDR<0.05 in both EoE vs healthy and post-dupilumab treatment vs baseline comparisons.

DESeq2 version 1.26.0 was used to perform differential expression analysis. Within the two arms (Placebo and dupilumab 300 mg QW) week 12 was compared to baseline. Genes were considered to be significantly modulated by treatment (dupilumab or placebo) if thresholds of a relative log change from baseline≥2, q-value 0.05 were reached, reflecting adjustment for multiple testing. Pearson correlations were calculated between: (i) the published gene changes in EoE (disease vs healthy), and (ii) gene changes after dupilumab treatment (post- vs pre-treatment).

### Results

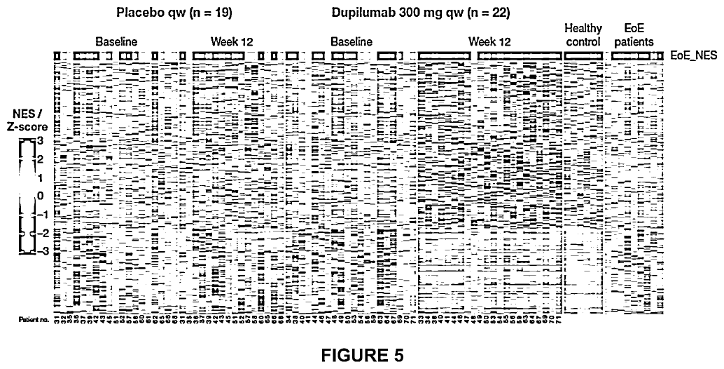

No genes were found to be differentially expressed within the placebo arm when comparing Week 12 to baseline (relative log change from baseline≥2, q≤0.05). At Week 12, treatment with dupilumab 300 mg QW vs baseline modulated (relative log change from baseline≥2, q≤0.05) expression of 1,302 genes, the DpxOme-EoE™, of which 513 were downregulated and 789 upregulated (FIG. 5). As the post-treatment results were highly similar across all three esophageal regions sampled, mean values are presented across all samples. The top 50 most upregulated and top 50 most downregulated genes in EoE were used to generate a normalized enrichment score (EoE-NES). Across genes, dupilumab treatment showed significantly lower EoE-NES (Wilcoxon rank sum test, P<5.0×10⁻⁸), with no significant changes seen in the placebo group. The 30 genes showing the highest changes in expression by dupilumab included those associated with type 2 inflammation, tissue remodeling/fibrosis, barrier function, and proliferation/differentiation (FIG. 6). Genes upregulated in the EoE transcriptome that were downregulated by dupilumab included ALOX15, CCL26, POSTN, NRXN1, and CCR3; genes downregulated in disease and upregulated by dupilumab included SPINK8 and DSG1.

When compared to the published EoE transcriptome vs healthy transcriptome, treatment with dupilumab (Week 12 vs baseline) normalized the transcriptome at Week 12. A strong, negative correlation was observed between the published EoE transcriptome (vs healthy) and the DpxOme-EoE™ (Week 12 vs. baseline) (Pearson correlation co-efficient: ρ=−0.872, P<1×10⁻⁶). There was a normalization trend for genes that did not meet the significance thresholds, and dupilumab also significantly modulated a number of genes which were not included in the published EoE transcriptome. Furthermore, many of the genes that did not overlap between the EoE and dupilumab signatures were not measurable in one or the other dataset. The main genes which are altered in EoE and were modified by dupilumab treatment included the following gene ontology (GO) groups: immune function/inflammation (e.g., interleukin-12 production, B cell mediated immunity, response to type 1 interferon), eosinophil migration remodeling (e.g., extracellular matrix disassembly), mast cell activation, and epithelial differentiation (e.g., keratinization and cornification).

At Week 12, dupilumab modulated type 2 inflammatory genes, eosinophil-associated genes, and genes associated with mast cell activation. These modulated genes included CCL26, MUC5B, CLC, IL1RL1, HDC, IL13, FCER1G, GATA2, and KIT. Dupilumab treatment reduced eosinophil tissue infiltration at Week 12, with similar effects on all three regions sampled. Changes observed in eosinophil-associated gene expression were consistent with the decrease in density of eosinophils observed in esophageal biopsies after dupilumab treatment.

At Week 12, dupilumab treatment also modulated genes associated with fibrosis, stroma remodeling, TGFβ and integrin signaling, such as collagen family genes and barrier-associated genes including DSG1, SPINK5, SPINK7, and SPINK8. Additional changes in expression were observed in type 1 inflammatory genes, published anti-IL-13 genes, and genes which were not modulated by the anti-IL-13 antibody QAX576.

To evaluate the relationship between gene expression profiles and total EoE-HSS grade score, eosinophil counts, and mucosal inflammatory and remodeling features (EoE-EREFS), correlation analyses were performed using the individual gene results, as well as NES. The DpxOme-EoE™ NES score was strongly correlated with histological severity (ρ=0.832, P<0.001), demonstrating a biological association of the molecular signature with the clinical measure. Additionally, the expression of several individual genes was also highly correlated. The single most highly correlated gene with the total EoE-HSS was CTSC (cathepsin C; ρ=0.826, P<0.001), a protease involved in activating other pro-inflammatory proteinases. This gene was also found to be highly correlated with eosinophil counts (ρ=0.783, P<0.001), together with other genes including CCL26, CCR3, ANO1, and SPINK8. Other correlations ranging from ρ=0.585 to 0.623 were observed with EoE-EREFS.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. The disclosures of all patents and non-patent literature cited herein are expressly incorporated in their entirety by reference.

TABLE 10
Sequence Listing

| SEQ | | |
|---|---|---|
| ID NO | Sequence (amino acid) | Description |
| 1 | EVQLVESGGGLEQPGGSLRLSCAGSGFTFRDYAMTWVRQAPGKGLEWVSSISGSG | Dupilumab HCVR amino |
| | GNTYYADSVKGRFTISRDNSKNTLYLQMNSLRAEDTAVYYCAKDRLSITIRPRYYGLD | acid sequence |
| | VWGQGTTVTVS | |
| 2 | DIVMTQSPLSLPVTPGEPASISCRSSQSLLYSIGYNYLDWYLQKSGQSPQLLIYLGSNR | Dupilumab LCVR amino |
| | ASGVPDRFSGSGSGTDFTLKISRVEAEDVGFYYCMQALQTPYTFGQGTKLEIK | acid sequence |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 amino |
| | | acid sequence |
| 7 | LGS | Dupilumab LCDR2 amino |
| | | acid sequence |
| 8 | MQALQTPYT | Dupilumab LCDR3 amino |
| | | acid sequence |
| 9 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab light chain |
| | ASGVPDRFSGSGSGTDFTLKISRVEAEDVGFYYCMQALQTPYTFGQGTKLEIKRTVAA | amino acid sequence |
| | PSVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSFNRGEC | |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGTLSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLQPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLQPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLQPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLQPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLQPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLQPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |

What is claimed is:

1. A method of treating, preventing, or ameliorating at least one symptom of eosinophilic esophagitis (EoE) in a subject≥12 years of age, the method comprising administering to the subject one or more doses of an interleukin-4 receptor (IL-4R) inhibitor, wherein prior to the onset of treatment the subject has a Dysphagia Symptom Questionnaire (DSQ) score≥10, and wherein the IL-4R inhibitor is an antibody or antigen-binding fragment thereof that binds IL-4Rα and comprises a heavy chain complementarity determining region (HCDR)1 comprising the amino acid sequence of SEQ ID NO:3, an HCDR2 comprising the amino acid sequence of SEQ ID NO:4, an HCDR3 comprising the amino acid sequence of SEQ ID NO:5, a light chain complementarity determining region (LCDR)1 comprising the amino acid sequence of SEQ ID NO:6, an LCDR2 comprising the amino acid sequence of SEQ ID NO:7, and an LCDR3 comprising the amino acid sequence of SEQ ID NO:8.

2. The method of claim 1, wherein the subject is an adult.

3. The method of claim 1, wherein the subject is an adolescent≥12 and <18 years of age.

4. The method of claim 1, wherein prior to the onset of treatment the subject has an intraepithelial eosinophilic infiltration peak cell count≥15 eos/hpf as measured by endoscopic biopsy in at least two of the proximal esophageal region, mid esophageal region, and distal esophageal region.

5. The method of claim 1, wherein the subject has a concomitant atopic disease.

6. The method of claim 5, wherein the concomitant atopic disease is a food allergy, atopic dermatitis, asthma, chronic rhinosinusitis, allergic rhinitis, or allergic conjunctivitis.

7. The method of claim 1, wherein the subject has eosinophilic gastroenteritis.

8. The method of claim 1, wherein the subject has a history of an average of at least two episodes of dysphagia per week for at least 4 weeks.

9. The method of claim 1, wherein the subject is unresponsive or inadequately responsive to treatment with a swallowed topical corticosteroid and/or a proton pump inhibitor (PPI).

10. The method of claim 1, wherein the IL-4R inhibitor comprises a heavy chain variable region (HCVR) comprising the amino acid sequence of SEQ ID NO:1 and a light chain variable region (LCVR) comprising the amino acid sequence of SEQ ID NO:2.

11. The method of claim 1, wherein the IL-4R inhibitor comprises a heavy chain comprising the amino acid sequence of SEQ ID NO:9 and a light chain comprising the amino acid sequence of SEQ ID NO:10.

12. The method of claim 1, wherein the IL-4R inhibitor is dupilumab or a bioequivalent thereof.

13. The method of claim 1, wherein the IL-4R inhibitor is administered at a dose of about 50 mg to about 600 mg.

14. The method of claim 13, wherein the IL-4R inhibitor is administered at a dose of about 300 mg.

15. The method of claim 13, wherein the IL-4R inhibitor is administered once a week or once every two weeks.

16. The method of claim 1, wherein the IL-4R inhibitor is administered in combination with a second therapeutic agent or therapy.

17. The method of claim 16, wherein the second therapeutic agent or therapy is an IL-1β inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a topical corticosteroid, an oral corticosteroid, a systemic corticosteroid, an inhaled corticosteroid, a glucocorticoid, a PPI, a decongestant, an antihistamine, a non-steroidal anti-inflammatory drug (NSAID), esophagus dilation, allergen removal, or diet management.

18. The method of claim 17, wherein the IL-4R inhibitor is administered in combination with a PPI.

19-27. (canceled)

28. A method of improving the ability to swallow food in a subject, the method comprising:
administering to a subject having eosinophilic esophagitis (EoE) one or more doses of an interleukin-4 receptor (IL-4R) inhibitor, wherein the IL-4R inhibitor is an antibody or antigen-binding fragment thereof that binds IL-4Rα and comprises a heavy chain complementarity determining region (HCDR)1 comprising the amino acid sequence of SEQ ID NO:3, an HCDR2 comprising the amino acid sequence of SEQ ID NO:4, an HCDR3 comprising the amino acid sequence of SEQ ID NO:5, a light chain complementarity determining region (LCDR)1 comprising the amino acid sequence of SEQ ID NO:6, an LCDR2 comprising the amino acid sequence of SEQ ID NO:7, and an LCDR3 comprising the amino acid sequence of SEQ ID NO:8.

29. The method of claim 28, wherein the subject is ≥12 years of age.

30. The method of claim 29, wherein the subject is an adult.

31. The method of claim 29, wherein the subject is an adolescent≥12 and <18 years of age.

32. The method of claim 28, wherein prior to the onset of treatment the subject has a Dysphagia Symptom Questionnaire (DSQ) score≥10.

33. The method of claim 28, wherein the subject has a concomitant atopic disease.

34. The method of claim 33, wherein the concomitant atopic disease is a food allergy, atopic dermatitis, asthma, chronic rhinosinusitis, allergic rhinitis, or allergic conjunctivitis.

35. The method of claim 28, wherein the subject has eosinophilic gastroenteritis.

36. The method of claim 28, wherein the subject has a history of an average of at least two episodes of dysphagia per week for at least 4 weeks.

37. The method of claim 28, wherein the subject is unresponsive or inadequately responsive to treatment with a swallowed topical corticosteroid and/or a proton pump inhibitor (PPI).

38. The method of claim 28, wherein the IL-4R inhibitor comprises a heavy chain variable region (HCVR) comprising the amino acid sequence of SEQ ID NO:1 and a light chain variable region (LCVR) comprising the amino acid sequence of SEQ ID NO:2.

39. The method of claim 28, wherein the IL-4R inhibitor comprises a heavy chain comprising the amino acid sequence of SEQ ID NO:9 and a light chain comprising the amino acid sequence of SEQ ID NO:10.

40. The method of claim 28, wherein the IL-4R inhibitor is dupilumab or a bioequivalent thereof.

41. The method of 28, wherein the IL-4R inhibitor is administered at a dose of about 50 mg to about 600 mg.

42. The method of claim 41, wherein the IL-4R inhibitor is administered at a dose of about 300 mg.

43. The method of claim 41, wherein the IL-4R inhibitor is administered once a week or once every two weeks.

44. The method of claim 28, wherein the IL-4R inhibitor is administered in combination with a second therapeutic agent or therapy.

45. The method of claim 44, wherein the second therapeutic agent or therapy is an IL-1β inhibitor, an IL-5 inhibitor, an IL-9 inhibitor, an IL-13 inhibitor, an IL-17 inhibitor, an IL-25 inhibitor, a TNFα inhibitor, an eotaxin-3 inhibitor, an IgE inhibitor, a prostaglandin D2 inhibitor, an immunosuppressant, a topical corticosteroid, an oral corticosteroid, a systemic corticosteroid, an inhaled corticosteroid, a glucocorticoid, a PPI, a decongestant, an antihistamine, a non-steroidal anti-inflammatory drug (NSAID), esophagus dilation, allergen removal, or diet management.

46. The method of claim 45, wherein the IL-4R inhibitor is administered in combination with a PPI.

47. (canceled)

48. (canceled)

49. The method of claim 1, wherein the IL-4R inhibitor is contained within a container selected from the group consisting of a glass vial, a syringe, a pen delivery device, and an autoinjector.

50. The method of claim 28, wherein the IL-4R inhibitor is contained within a container selected from the group consisting of a glass vial, a syringe, a pen delivery device, and an autoinjector.
